(19) United States
(12) Patent Application Publication
(10) Pub. No.: US 2025/0205051 A1
(43) Pub. Date: Jun. 26, 2025

(54) Intravascular Delivery System

(71) Applicant: Cephea Valve Technologies, Inc., Abbott Park, IL (US)

(72) Inventors: Hugo A. Cobar, Sunnyvale, CA (US); Randolf Von Oepen, Aptos, CA (US)

(73) Assignee: Cephea Valve Technologies, Inc., Abbott Park, IL (US)

(21) Appl. No.: 19/073,582

(22) Filed: Mar. 7, 2025

Related U.S. Application Data
(63) Continuation of application No. PCT/US2023/036501, filed on Oct. 31, 2023.
(60) Provisional application No. 63/382,012, filed on Nov. 2, 2022.

(51) Int. Cl. A61F 2/24; A61M 25/00; A61M 25/10

(52) U.S. Cl. CPC A61F 2/2436; A61M 25/0045; A61M 25/0052; A61M 25/0054; A61M 25/1002; A61M 2025/0004; A61M 2025/1043; A61M 2205/0216

(57) ABSTRACT

A delivery system for delivering an implantable medical device, such as a prosthetic heart valve, intravascularly to a target site, such as a heart valve annulus in a patient. The system includes a catheter assembly consisting of a plurality of concentrically arranged catheters positioned within an outer sheath and a handle assembly that includes a plurality of controls that selectively translate the catheters relative to one another to deploy the medical device from the outer sheath. An optional inflatable balloon on the catheter assembly helps to visualize the position of the medical device prior to deployment. The delivery system accommodates both the deployment of the medical device as well as re-sheathing should that become necessary.

### CROSS-REFERENCE TO RELATED APPLICATIONS

The present application is a continuation of International Application No. PCT/US2023/036501 filed Oct. 31, 2023, which claims the benefit of the filing date of U.S. Provisional Patent Application No. 63/382,012 filed Nov. 2, 2022, the disclosures of which are both hereby incorporated by reference in their entirety as if fully set forth herein.

### BACKGROUND OF THE DISCLOSURE

The present disclosure relates generally to devices, systems and methods for delivering an interventional device into a patient for implantation. More particularly, the present disclosure relates to devices, systems and methods for transseptal delivery of a collapsible prosthetic heart valve to a native mitral valve annulus, and to the deployment of the prosthetic heart valve at the native mitral valve annulus. Interventional medical devices that are collapsible to a relatively small circumferential size can be delivered into a patient less invasively than devices that are not collapsible. For example, a collapsible prosthetic heart valve may be delivered into a patient via a tube-like delivery apparatus such as a catheter, a trocar, a laparoscopic instrument, or the like. This collapsibility can avoid the need for a more invasive procedure such as full open-chest, open-heart surgery, thereby reducing the risks, costs and time associated with an open-heart surgical procedure.

Collapsible prosthetic heart valves typically take the form of a valve structure mounted on a stent. There are two types of stents on which the valve structures are ordinarily mounted: a self-expanding stent and a balloon-expandable stent. Valves with self-expanding stents generally are first collapsed or crimped to reduce their circumferential size, and then loaded into a delivery apparatus. Valves with balloon-expandable stents generally are crimped around a deflated balloon that is mounted to a delivery apparatus.

When a collapsed prosthetic valve has reached the desired implantation site in the patient (e.g., at or near the annulus of the patient's heart valve that is to be replaced by the prosthetic valve), the prosthetic valve can be deployed or released from the delivery apparatus. For balloon-expandable valves, this generally involves assuring the proper location of the valve, and then expanding the balloon positioned within the valve stent. For self-expanding valves, on the other hand, the stent automatically expands as the sheath covering the valve is withdrawn.

There are many considerations in properly deploying a prosthetic heart valve in the native valve annulus. The prosthetic valve should be placed at the same or very nearly the same angle as the native valve. A valve that is off axis could cause turbulent blood flow and/or potential paravalvular leaks. Also, the prosthetic valve should be implanted so that its center aligns with the center of the native valve. Off-center deployment or implantation of the prosthetic valve could interfere with neighboring valves or the heart's conduction system. Finally, the prosthetic heart valve should be implanted at the proper depth within the native valve annulus, also so as to not interfere with the heart's conduction system.

A safe, accurate and efficient delivery system and method for a prosthetic heart valve that addresses some or all of the foregoing concerns is described herein.

### BRIEF SUMMARY OF THE INVENTION

The present invention provides multiple embodiments of a delivery system for delivering a medical device to a targeted anatomical site within a patient. One embodiment of the delivery system includes a catheter assembly, including an outer sheath having a proximal end and a distal end, and a valve cover at the distal end of the outer sheath. The valve cover has a proximal end, a distal end and a size and a shape for housing the medical device in a collapsed condition. A balloon is disposed circumferentially about an exterior surface of the valve cover between its proximal end and distal end, the balloon having an inflated condition and a deflated condition. The catheter assembly further includes a plurality of catheters coaxially arranged within the outer sheath, some of which are slidable in proximal and distal directions relative to the outer sheath.

Another embodiment of the delivery system includes a catheter assembly extending in a longitudinal direction. The catheter assembly includes an outer sheath having a first portion and a second portion that is more flexible than the first portion. The second portion has a coiled layer and a braided sleeve disposed around the coiled layer. The coiled layer has a proximal end and a distal end, and the braided sleeve has a proximal end connected to the first portion of the outer sheath and a distal end connected to the distal end of the coiled layer. A hypotube is disposed within the first portion of the outer sheath. A distal end of the hypotube is connected to the proximal end of the coiled layer, and a valve cover is connected to the distal end of the coiled layer. The valve cover has a proximal end, a distal end and a size and a shape for housing the medical device in a collapsed condition. The catheter assembly further includes a plurality of catheters coaxially arranged within the outer sheath and slidable in proximal and distal directions relative to the outer sheath.

A further embodiment of the delivery system includes a catheter assembly, including an outer sheath extending in a longitudinal direction and having a first portion and a second portion that is more flexible than the first portion. The second portion has a plurality of rings arranged adjacent one another in a stack, the stack having a proximal end and a distal end, the proximal end being connected to a distal end of the first portion. A plurality of filaments extend through each of the plurality of rings, each filament having a distal end connected to the distal end of the ring stack and a proximal end connected the first portion of the outer sheath. A valve cover at the distal end of the ring stack has a size and a shape for housing the medical device in a collapsed condition. The catheter assembly further includes a plurality of catheters coaxially arranged within the outer sheath and slidable in proximal and distal directions relative to the outer sheath.

Yet another embodiment of the delivery system includes a catheter assembly having a longitudinal axis. The catheter assembly includes an outer sheath having a proximal end and a distal end, and a valve cover at the distal end of the outer sheath. The valve cover has a proximal end, a distal end, and a size and a shape for housing the medical device in the collapsed condition. A balloon is disposed circumferentially about an exterior surface of the valve cover between its proximal end and distal end, the balloon having an inflated condition and a deflated condition. An extension catheter is disposed within the outer sheath and is slidable in proximal and distal directions relative to the outer sheath. A suture catheter is disposed within the extension catheter and is slidable in the proximal and distal directions relative to the extension catheter. The suture catheter is adapted to maintain a connection to the medical device until the medical device is deployed. The delivery system also includes a base, and a handle assembly connected to the base for controlling movement of the outer sheath, the extension catheter and the suture catheter relative to one another. The handle assembly includes an outer sheath actuator operatively connected to the outer sheath and operable to move the outer sheath in the proximal and distal directions relative to the base. The handle assembly also includes an extension catheter holder connected to the extension catheter, a suture catheter control connected to the suture catheter proximally of the extension catheter holder, and a locking mechanism. The locking mechanism has a locked condition in which the position of the suture catheter control is fixed relative to the extension catheter holder so that the suture catheter and the extension catheter move together in the proximal and distal directions relative to the outer sheath. The locking mechanism also has a release condition in which the suture catheter control is movable in the proximal and distal directions relative to the extension catheter holder.

A still further embodiment of the delivery system includes a catheter assembly extending in a longitudinal direction, the catheter assembly including an outer sheath having a first portion and a second portion that is more flexible than the first portion. The second portion has a coiled layer and a braided sleeve disposed around the coiled layer. The coiled layer has a proximal end and a distal end, and the braided sleeve has a proximal end connected to the first portion of the outer sheath and a distal end connected to the distal end of the coiled layer. A hypotube is disposed within the first portion of the outer sheath. A distal end of the hypotube is connected to the proximal end of the coiled layer. A valve cover is connected to the distal end of the coiled layer, the valve cover having a proximal end, a distal end, and a size and shape for housing the medical device in a collapsed condition. An extension catheter is disposed within the outer sheath and is slidable in proximal and distal directions relative to the outer sheath. A suture catheter is disposed within the extension catheter and is slidable in the proximal and distal directions relative to the extension catheter, the suture catheter being adapted to maintain a connection to the medical device until deployment of the medical device. The delivery system also includes a base and a handle assembly connected to the base for controlling movement of the outer sheath, the extension catheter and the suture catheter relative to one another. The handle assembly includes an outer sheath actuator operatively connected to the outer sheath and operable to move the outer sheath in the proximal and distal directions relative to the base. The handle assembly also includes an extension catheter holder connected to the extension catheter, a suture catheter control connected to the suture catheter proximally of the extension catheter holder, and a locking mechanism. The locking mechanism has a locked condition in which the position of the suture catheter control is fixed relative to the extension catheter holder so that the suture catheter and the extension catheter move together in the proximal and distal directions relative to the outer sheath. The locking mechanism also has a release condition in which the suture catheter control is movable in the proximal and distal directions relative to the extension catheter holder.

Yet another embodiment of the delivery system includes a catheter assembly extending in a longitudinal direction, the catheter assembly including an outer sheath having a first portion and a second portion that is more flexible than the first portion. The second portion has a plurality of rings that are arranged adjacent one another in a stack. The stack has a proximal end and a distal end, the proximal end being connected to the distal end of the first portion. A plurality of filaments extend through each of the plurality of rings. Each filament has a distal end connected to the distal end of the ring stack and a proximal end connected to the first portion of the outer sheath. A valve cover is connected to the distal end of the coiled layer, the valve cover having a proximal end, a distal end, and a size and shape for housing the medical device in a collapsed condition. An extension catheter is disposed within the outer sheath and is slidable in proximal and distal directions relative to the outer sheath. A suture catheter is disposed within the extension catheter and is slidable in the proximal and distal directions relative to the extension catheter, the suture catheter being adapted to maintain a connection to the medical device until deployment of the medical device. The delivery system also includes a base and a handle assembly connected to the base for controlling movement of the outer sheath, the extension catheter and the suture catheter relative to one another. The handle assembly includes an outer sheath actuator operatively connected to the outer sheath and operable to move the outer sheath in the proximal and distal directions relative to the base. The handle assembly also includes an extension catheter holder connected to the extension catheter, a suture catheter control connected to the suture catheter proximally of the extension catheter holder, and a locking mechanism. The locking mechanism has a locked condition in which the position of the suture catheter control is fixed relative to the extension catheter holder so that the suture catheter and the extension catheter move together in the proximal and distal directions relative to the outer sheath. The locking mechanism also has a release condition in which the suture catheter control is movable in the proximal and distal directions relative to the extension catheter holder.

### BRIEF DESCRIPTION OF THE DRAWINGS

A more complete appreciation of the subject matter of the present disclosure and the various advantages thereof can be realized by reference to the following detailed description in which reference is made to the accompanying drawings in which:

FIG. 1A is an illustrative prosthetic heart valve according to the prior art for use in conjunction with the delivery system of the present invention;

FIG. 1B is a perspective view of the frame structure of the prosthetic heart valve of FIG. 1A;

FIG. 1C is an enlarged view of a portion of the frame structure of FIG. 1B;

FIG. 2 is a side view of a delivery system configured for delivering, positioning and deploying a prosthetic heart valve, including a handle assembly and a catheter assembly;

FIG. 3A is a transverse cross-sectional view of the catheter assembly of FIG. 2, showing the various components thereof;

FIG. 3B is a longitudinal cross-section of the catheter assembly of FIG. 2, showing the nested arrangement of the various components thereof;

FIG. 4 is a schematic cutaway view of the heart, showing an exemplary approach for delivering the prosthetic heart valve to the mitral valve annulus;

FIG. 5 is a side view of an outer sheath of the catheter assembly, showing the nosecone in a closed position at the distal end thereof;

FIG. 6 is an enlarged view of the distal end of the outer sheath and valve cover shown in FIG. 5;

FIG. 7 is a longitudinal cross-section of the distal end of the outer sheath and the valve cover shown in FIG. 6;

FIG. 8 is an enlarged cutaway view of a highly flexible portion of the outer sheath;

FIG. 9 is a side view of a steering catheter of the catheter assembly, showing various sections thereof;

FIG. 10A is a perspective view of a tip ring that may be used at the distal end of the steering catheter;

FIG. 10B is a longitudinal cross-section of the tip ring of FIG. 10A connected to the distal end of the steering catheter;

FIG. 11 is an enlarged partial view of the distal section of the steering catheter of FIG. 9 forming a compound curve shape to enable proper positioning of the catheter assembly relative to the mitral valve annulus;

FIG. 12A is an enlarged side view illustrating an embodiment of a cut pattern that may be formed in the distal section of the suture catheter and/or in other components of the catheter assembly to provide a gradient bending profile;

FIG. 12B is an enlarged partial view illustrating bending of the distal section of the steering catheter;

FIG. 13A is a side view of an extension catheter of the catheter assembly with a distal can structure at its distal end;

FIG. 13B is an enlarged partial cross-section of the distal section of the extension catheter and distal can structure of FIG. 13A;

FIG. 13C is an enlarged highly schematic view illustrating the tri-coil structure in the distal section of the extension catheter;

FIG. 13D is an enlarged partial view of the extension catheter of FIG. 13A, showing the connection of the proximal end to an extension catheter holder;

FIG. 14A is a side view of a suture catheter of the catheter assembly;

FIG. 14B is a longitudinal cross-section of the suture catheter of FIG. 14A;

FIG. 14C is an enlarged longitudinal cross-section of the distal end of the suture catheter of FIG. 14A;

FIG. 14D is an enlarged highly schematic view of a portion of an exemplary suture catheter with a distal suture ring and a plurality of tethers attached thereto;

FIG. 15A is a perspective view of a distal suture ring for use with the suture catheter of FIG. 14A;

FIG. 15B is a perspective view showing tethers attached to the distal suture ring of FIG. 15A;

FIG. 15C is a side view of a prosthetic mitral valve assembly consisting of the distal suture ring and tethers of FIG. 15B attached to a prosthetic mitral valve, which is held by a portion of a packaging assembly;

FIG. 16A is a side view of a nosecone catheter and nosecone of the catheter assembly;

FIG. 16B is a longitudinal cross-section of the nosecone of FIG. 16A;

FIG. 16C is a perspective view of an insert for the nosecone of FIG. 16A;

FIG. 17A is a side view of a guidewire for use with the catheter assembly, showing the pigtail distal end;

FIG. 17B is a side view of the guidewire of FIG. 17A shown in a straightened configuration;

FIG. 18 is a highly schematic longitudinal cross-section of a portion of a valve cover according to another embodiment, showing a balloon in deflated and inflated conditions;

FIG. 19 is an enlarged perspective view of the handle assembly shown in FIG. 2;

FIG. 20 is an enlarged side view of a portion of the handle assembly showing the controls for the suture catheter and nosecone catheter;

FIG. 21 is an exploded view of the release mechanism for the suture catheter control (as well as for the nosecone catheter control);

FIG. 22A and 22B illustrate the advancement and retraction of the nosecone in response to movement of the nosecone catheter control;

FIG. 23A is a side view showing the delivery system mounted to a stabilizer;

FIG. 23B is a perspective view showing the delivery system mounted to the stabilizer of FIG. 23A;

FIG. 23C is a perspective view showing the delivery system mounted to the stabilizer of FIG. 23A in an inclined position;

FIG. 24 is a side view of a loading funnel attached to the distal end of the valve cover;

FIGS. 25A through 25F illustrate the deployment and release of a prosthetic mitral valve at the mitral valve annulus;

FIG. 26A is a longitudinal cross-section of the distal end of an alternate embodiment of an outer sheath in which the unsheathing and re-sheathing forces are decoupled;

FIG. 26B is a longitudinal cross-section of the distal end of the outer sheath of FIG. 26A, showing the coils of the coiled layer in stacked arrangement;

FIG. 27A is a longitudinal cross-section of the distal end of an alternate embodiment of an outer sheath having stacked rings held in alignment by filaments;

FIG. 27B is a transverse cross-section though one of the rings shown in FIG. 27A, showing the tapered apertures therein; and

FIG. 28 is a longitudinal cross-section of the distal end of the outer sheath of FIG. 27A, showing the connection of the filaments to the proximal portion of the outer sheath.

### DETAILED DESCRIPTION

As used herein, the term “inflow end,” when used in connection with a prosthetic heart valve, refers to the end of the heart valve through which blood enters when the heart valve is functioning as intended, whereas the term “outflow end,” when used in connection with a prosthetic heart valve, refers to the end of the heart valve through which blood exits when the heart valve is functioning as intended. For a prosthetic mitral valve, the inflow end is closest to the left atrium when the heart valve is implanted in a patient, and the outflow end is closest to the left ventricle when the heart valve is implanted in a patient. Further, when used herein in connection with a delivery device, the terms “proximal” and “distal” are to be taken as relative to a user operating the device in an intended manner. “Proximal” is to be understood as relatively close to the user and “distal” is to be understood as relatively farther away from the user. Also as used herein, the terms “substantially,” “generally,” and “about” are intended to mean that slight deviations from absolute are included within the scope of term so modified.

In the description which follows, a delivery system and the components thereof are described in connection with the delivery, positioning and deployment of a prosthetic mitral valve at the native mitral valve annulus. However, it is to be understood that the delivery system and components described also may be used to deliver, position and deploy prosthetic replacements for other cardiac valves, such as the aortic valve, the pulmonary valve and the tricuspid valve, as well as other medical devices. Exemplary prosthetic heart valves that can be used with the delivery system described herein include the expandable prosthetic heart valves described in U.S. Patent Publication No. 2016/0158000; in U.S. Pat. No 8,870,948; and in PCT Publication No. WO 2016/183526, the disclosures of all of which are hereby incorporated by reference herein.

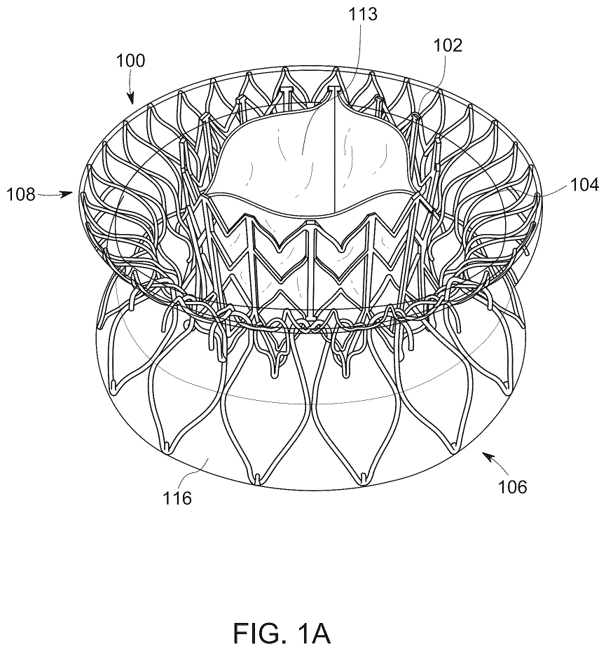

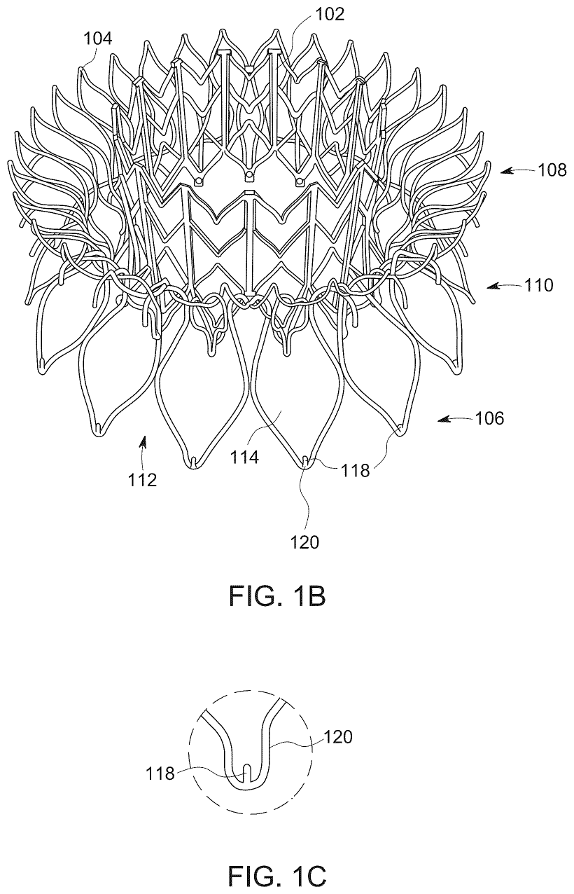

FIG. 1A is a perspective view of an exemplary prosthetic heart valve 100 that may be delivered, positioned and deployed by the delivery system disclosed herein. Prosthetic heart valve 100 may be a prosthetic mitral valve having an expandable and collapsible frame structure that includes an inner strut frame 102 surrounded by an outer anchor assembly 104, as shown in FIG. 1B. Anchor assembly 104, shown in an expanded state, includes an atrial anchor 106 configured to be positioned on the atrial side of the native mitral valve annulus, a ventricular anchor 108 configured to be positioned on the ventricular side of the native mitral valve annulus, and a central portion 110 positioned axially between the atrial anchor and the ventricular anchor. Anchor assembly 104 may have an hourglass shape in the expanded state in that each of atrial anchor 106 and ventricular anchor 108 flares radially outward of central portion 110, such that the central portion defines a waist between the atrial anchor and the ventricular anchor. Strut frame 102 may be positioned radially inward of anchor assembly 104 and may be formed of a plurality of interconnected struts. The radially inner surface of strut frame 102 defines the perimeter of a central opening 112, which enables blood to flow through prosthetic mitral valve 100. Exemplary replacement heart valves are described in U.S. Pat. No. 10,470,881, filed on Feb. 28, 2018 and entitled Replacement Mitral Valves, the disclosure of which is hereby incorporated by reference herein in its entirety.

Prosthetic mitral valve 100 includes one or more leaflets 113 that may be secured to strut frame 102 and disposed at least partially in central opening 112. Leaflets 113 are configured to coapt with one another to control blood flow through the prosthetic mitral valve, allowing blood to flow from atrial anchor 106 at the inflow end of the heart valve toward ventricular anchor 108 at the outflow end of the heart valve (the antegrade direction), but substantially blocking blood from flowing in the opposite (retrograde) direction. In some embodiments, one or more skirts or cuffs 116 may partially or fully cover inner and/or outer surfaces of anchor assembly 104 and/or strut frame 102. Such skirts or cuffs may be formed from fabric and/or tissue materials, for example.

Both the atrial anchor 106 and the ventricular anchor 108 of anchor assembly 104 include a plurality of petals 114 that are joined to one another around the circumference of the anchor assembly. When prosthetic mitral valve 100 is in a fully expanded state, the petals 114 on both atrial anchor 106 and ventricular anchor 108 are fully extended radially outward, as shown in FIG. 1B. Prosthetic mitral valve 100 is naturally in an expanded state when no force is applied to petals 114. The petals 114 of anchor assembly 104 may be configured to collapse and/or to reduce the outer diameter of the frame structure when the prosthetic heart valve is loaded into a delivery device. When prosthetic mitral valve 100 is in a collapsed state, the petals 114 on both atrial anchor 106 and ventricular anchor 108 are at least partially collapsed radially inward. Prosthetic mitral valve 100 may be placed in the collapsed state by applying pressure to petals 114 in a radially inward direction.

The petals 114 on atrial anchor 106 or ventricular anchor 108 may include a pin 118 or other attachment member to which tether loops may be connected, as will be described below. Pins 118 may be attached to or formed on some or all of the petals 114 on atrial anchor 106 and/or ventricular anchor 108 and are sized and shaped so that the tether loops remain attached when under tension but are released from the frame structure after the deployment of prosthetic valve 100 within the patient. Pins 118 may be provided, for example, at the apex 120 of each petal 114 on atrial anchor 106, as shown in FIG. IC. However, this need not be the case and pins 118 may be provided on less than all of the petals 114 of atrial anchor 106, on some or all of the petals of ventricular anchor 108, or at other locations on anchor assembly 104.

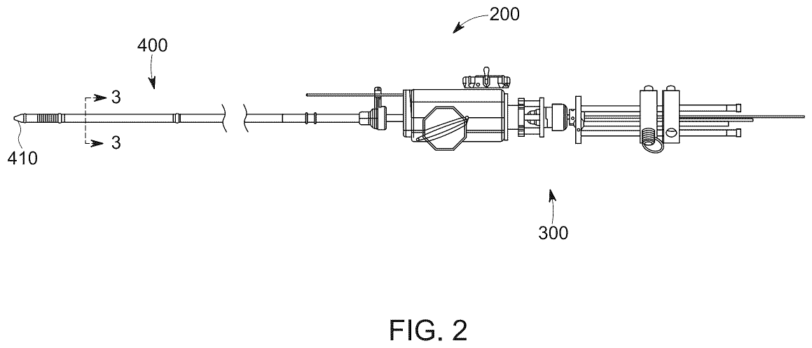

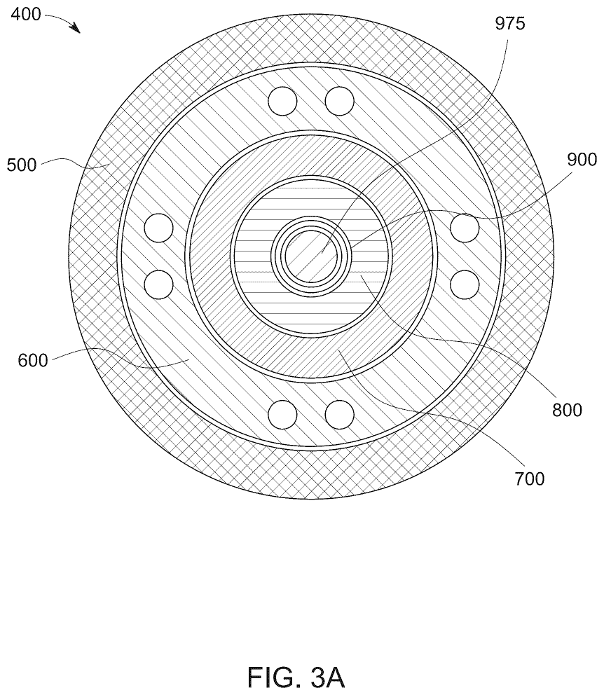

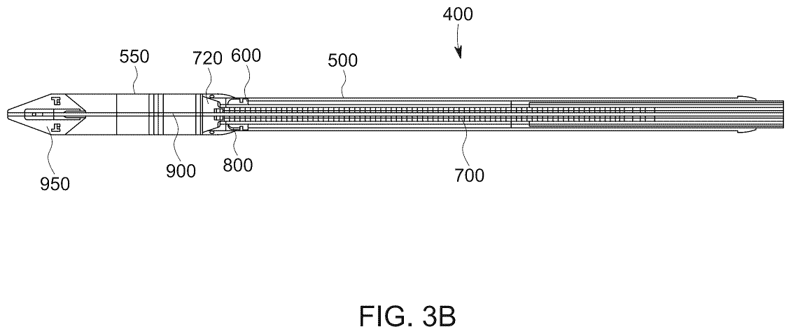

FIG. 2 illustrates an exemplary embodiment of a delivery system 200 for delivering and deploying a prosthetic heart valve at a target location within the patient's heart. Delivery system 200 generally includes a handle assembly 300 and a catheter assembly 400. Catheter assembly 400 extends from a proximal end coupled to handle assembly 300 to an atraumatic tip 410 at a distal end and includes a plurality of catheter and/or hypotube components that are longitudinally slidable relative to one another and that provide different functionality during operation of delivery system 200 to enable effective delivery and deployment of a prosthetic heart valve, such as prosthetic mitral valve 100. As shown in FIG. 3A, which is a cross-sectional view of catheter assembly 400 taken along line 3-3 of FIG. 2, these components include an outer sheath 500, a steering catheter 600, an extension catheter 700, a suture catheter 800, and a nosecone catheter 900, all arranged in a concentric nested relationship. The arrangement of these components, as well as valve cover 550 and nosecone 950, is shown in the longitudinal cross-section of catheter assembly 400 shown in FIG. 3B. As illustrated in the figures, nosecone catheter 900 has a lumen sized to receive a guidewire 975 therein. Each of these components is described in detail below.

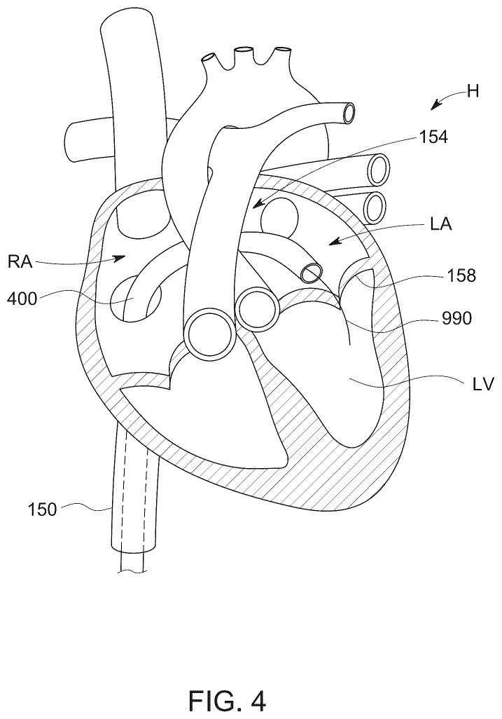

FIG. 4 is a schematic representation of a patient's heart H and a delivery route that may be followed by catheter assembly 400 to reach the native mitral valve annulus 158. Using a transfemoral approach, catheter assembly 400 may be inserted into the patient's femoral vein and advanced through the inferior vena cava 150 to the right atrium RA. In a subsequent transseptal procedure, catheter assembly 400 is advanced through a puncture in intra-atrial septum 154 into the left atrium LA.

In other implementations, such as for procedures associated with a tricuspid valve, catheter assembly 400 may be advanced through the inferior vena cava 150 and into the right atrium RA, where it may then be positioned and used to perform the procedure related to the tricuspid valve. While many of the examples described herein relate to delivery to the native mitral valve annulus, one or more embodiments may be utilized in other cardiac procedures, including those involving the tricuspid valve or other cardiac valves.

Although one preferred method for accessing a targeted cardiac valve annulus is a transfemoral approach, it will be understood that the embodiments described herein may also be utilized where alternative approaches are used. For example, embodiments described herein may be utilized in a transjugular approach, transapical approach, transradial approach or other suitable approaches to the targeted anatomy. For procedures related to the mitral valve or tricuspid valve, the delivery of the prosthetic heart valve or other medical device is preferably carried out from an atrial aspect (i.e., with the distal end of catheter assembly 400 positioned within the atrium superior to the targeted valve). The illustrated embodiments are shown from such an atrial aspect. However, it will be understood that the delivery of the medical devices described herein may also be carried out from a ventricular aspect.

Additional details regarding delivery systems and devices that may be utilized in conjunction with the components and features described herein are described in U.S. Patent Publication Nos. 2018/0028177A1, 2018/0092744A1, and 2020/0155804, the disclosures of which are hereby incorporated by reference herein.

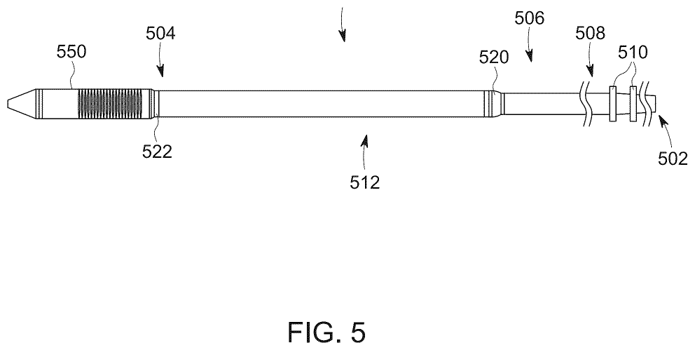

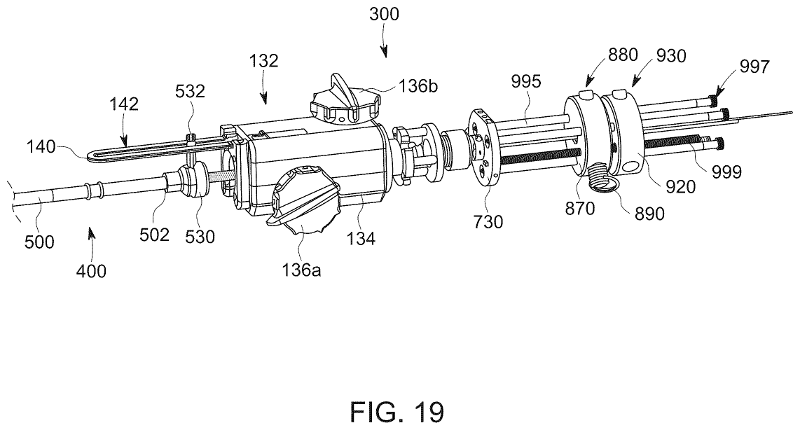

As noted, the outer component of catheter assembly 400 is outer sheath 500, shown in FIGS. 5-7. Outer sheath 500 extends from a proximal end 502 to a distal end 504. A proximal portion 506 of outer sheath 500 may be formed from a stainless steel hypotube having a plurality of slits (not shown) laser cut in an interrupted spiral pattern along a majority of its length. The slits preferably are cut parallel to one another in an alternating staggered pattern. For example, four slits may be spaced apart from one another in a single ring around the circumference of proximal portion 506. In the next adjacent ring, the slits may be angularly offset from the slits in the first ring by about 45°. In the next adjacent ring, the slits may again be angularly offset from the slits in the previous ring by 45° so that the slits are aligned in the longitudinal direction of outer sheath 500 with the slits in the first ring. This pattern of slits may continue along the length of proximal portion 506. The slits begin at a spaced distance from the proximal end 502 of outer sheath 500, leaving an uncut portion 508 that enables the proximal end of the outer sheath to be fixedly connected, such as by adhesive, to a hemostasis valve 530 (see, for example, FIG. 19) with a leakproof seal. A pair of fixed rings 510 may be spaced apart from one another around the outside of uncut portion 508. Rings 510 enable outer sheath 500 to be held in place as other components of catheter assembly 400 are advanced or retracted, and also enable the outer sheath itself to be retracted for deploying prosthetic mitral valve 100, as will be discussed further below. Although outer sheath 500 is described as formed from a stainless steel hypotube, that need not be the case. Outer sheath 500 may alternatively be formed from other metals or metal alloys, including titanium or tantalum, from biologically stable polymers, from biologically stable composites, from a braided polymer shaft (as long as the shaft can transmit the axial forces needed to unsheathe a valve), and the like.

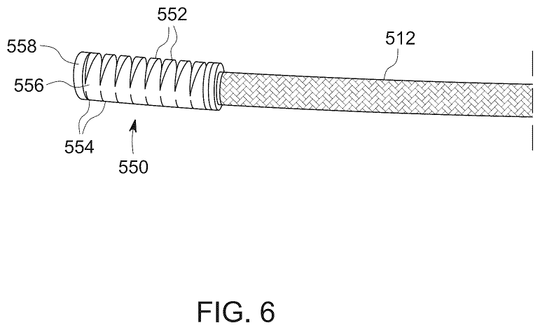

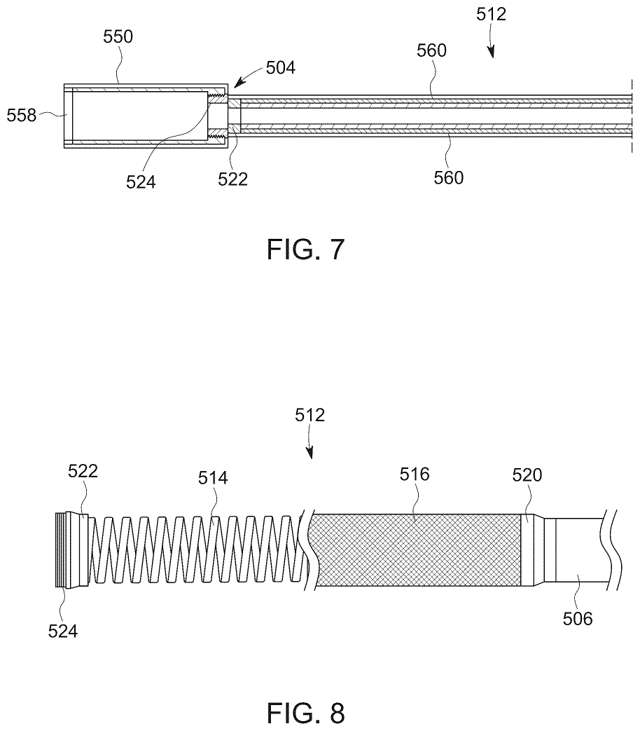

FIGS. 6 and 7 illustrate a distal portion of outer sheath 500 and a connected valve cover 550. The distal portion of outer sheath 500 includes a highly flexible portion 512 extending from proximal portion 506 to the distal end 504 of the outer sheath and having a sufficient length to surround and extend along that portion of catheter assembly 400 that is designed to bend and reorient to navigate through a patient's vasculature and heart to reach mitral valve annulus 158 for deployment of the prosthetic heart valve. In some embodiments, flexible portion 512 may have an inner coiled layer 514 formed from a coiled wire and an outer braided sleeve 516 covering the coiled layer (sometimes collectively referred to as “coil/braid portion 514/516”), as shown in FIG. 8. Coiled layer 514 is formed so that there are spaces between adjacent turns of the coil. In some embodiments, the spaces between adjacent turns of the coil are greater than the cross-section of the wire forming the coil. In such embodiments, longitudinally compressing the coil to eliminate the spaces between adjacent turns can reduce the overall length of coiled layer 514 by about 50% to about 70% or more of its original length. Braided sleeve 516 may be fixedly connected at its proximal and distal ends to coiled layer 514 by soldering through the filaments of the braid. Braided sleeve 516 may also be connected to coiled layer 514 by adding a small ring over the proximal and distal ends of the coil/braid portion 514/516 and laser welding all three components together simultaneously. The resultant coil/braid portion 514/516 exhibits a high degree of flexibility.

In one embodiment, shown in FIG. 8, coil/braid portion 514/516 may be joined to the proximal portion 506 of outer sheath 500 by first laser welding both ends of coiled layer 514 to separate sections of hypotube. Braided sleeve 516 may then be assembled over coiled layer 514 and soldered in place at the ends of the coiled layer. The hypotube section at the proximal end of coil/braid portion 514/516 may then be cut, leaving a short section of a solid ring 520 that may be laser welded to the proximal section 506 of outer sheath 500. The hypotube section at the distal end of coil/braid portion 514/516 may also be cut, leaving a short section of a solid ring 522. Another stainless-steel ring 524 that has a slightly larger diameter and that is externally threaded may then be laser welded to ring 522. The external threads provide for a threaded connection of valve cover 550 to coil/braid portion 514/516, and the slightly larger diameter of ring 524 enables the connection to be made without any exposed edges. A threaded connection enables valve cover 550 to be removably connected to outer sheath 500. Additionally, a threaded connection facilitates the joining of dissimilar metals, such as a titanium valve cover 550 to a stainless-steel outer sheath 500. However, when possible, the valve cover alternatively may be laser welded to the outer sheath.

As outer sheath 500 is retracted to deploy the prosthetic heart valve, internal friction between collapsed prosthetic mitral valve 100 and the valve cover 550 in which it is housed will inhibit the retraction of coiled layer 514, causing the individual coils to slightly separate farther from one another and braided sleeve 516 to lengthen. As braided sleeve 516 lengthens, its diameter will collapse around coiled layer 514, much like a Chinese finger trap, preventing further lengthening of coil layer 514 while still retaining the flexibility of flexible portion 512. Further, the collapse of braided sleeve 516 around coiled layer 514 keeps flexible portion 512 round when it is bent, preventing it from assuming an oval shape that could make it difficult to retract outer sheath 500 to deploy the prosthetic heart valve.

Valve cover 550 defines a compartment for housing prosthetic mitral valve 100 in a compressed, pre-deployed state during intravascular delivery of the prosthetic valve to the targeted cardiac site. Valve cover 550 may be formed by milling a solid rod of a hard, lightweight metal, such as grade 5 titanium, to form a generally cylindrical very thin-walled tube having an inner diameter and length sized to receive the prosthetic mitral valve in a collapsed condition. The titanium rod may be milled (for example, turned on a lathe) to a wall thickness of between about 0.10 mm (or thinner) and about 0.50 mm, or between about 0.30 mm and about 0.40 mm. A valve cover 550 having a wall thickness within the foregoing ranges may be sufficiently radiolucent to enable x-ray visualization of prosthetic mitral valve 100 when loaded therein during a procedure. Moreover, valve cover 550 may be formed with a thicker wall (for example, about 0.30 mm) in some regions and a thinner wall (for example, about 0.10 mm) in other regions to ensure good visibility of the prosthetic mitral valve held within the valve cover. A series of internal threads (not shown) may be cut at the proximal end of valve cover 550 for engagement with the external threads of ring 524 at the distal end of outer sheath 500.

Titanium is sufficiently inert that it will not interact with or contaminate, or is less likely to interact with or contaminate, the nitinol forming the frame 102 of prosthetic mitral valve 100 as the valve is retracted into and held within valve cover 550. A series of V-shaped cuts 552 may be laser cut along one side of valve cover 550, and a series of slits 554 may be laser cut along the diametrically opposed side of the valve cover, leaving a pair of continuous longitudinal spines 556 along opposite sides of the valve cover. Cuts 552 and slits 554 may be formed by a short-pulse laser that sublimes the metal, thereby avoiding any remelt in the interior of valve cover 550. Additionally, on the last pass of laser cutting, the laser beam may be defocused to smooth the edges of cuts 552 and slits 554. The series of cuts 552 and slits 554 enable valve cover 550 to bend in a single plane. As valve cover 550 bends, cuts 552 will collapse, while slits 554 will open. Preferably, cuts 552 and slits 554 are sufficient to enable valve cover 550 to bend about 75° or more; preferably, about 90° or more, or about 150° or more, so that the valve cover can be pulled over the distal bend of the steering catheter as it is retracted during deployment of prosthetic mitral valve 100. Cuts 552 and slits 554 are not formed along a distal section of valve cover 550 so as to not interfere with the protruding tines of the prosthetic mitral valve 100 as the prosthetic valve is retracted into the valve cover. After the formation of cuts 552 and slits 554, valve cover 550 may be deburred, preferably using an electro-polishing process or micro-blasting process followed by electro-polishing, to soften any sharp edges and remove any extraneous metal. The foregoing describes the use of titanium to form valve cover 550 since titanium has excellent biocompatibility, excellent mechanical properties and there is little corrosion potential between titanium and the nitinol used to form prosthetic mitral valve 100. However, other sufficiently strong and inert metals and metal alloys may also be used to form the valve cover.

The spines 556 formed along opposite sides of valve cover 550 between cuts 552 and slits 554 provide the valve cover with sufficient tensile strength to withstand the retraction of outer sheath 500 during deployment of a prosthetic heart valve. The distal end of valve cover 550 may have a number of external threads (not shown) to attach a loading funnel (described below) to the valve cover. After prosthetic mitral valve 100 has been loaded into valve cover 550 and the funnel has been removed, a tantalum ring 558 may be threaded onto the distal end of the valve cover to cover the threads and ensure a smooth surface. When ring 558 is made from a very radiopaque material like tantalum, platinum iridium, gold or other materials with a high atomic number, the ring helps the user locate the end of valve cover 550 under x-ray imaging to assure the proper location and orientation of the valve cover for deployment of the prosthetic valve. Rather than incorporating threads at the proximal end of valve cover 550 for removable connection to outer sheath 500 and at the distal end of the valve cover for the removable connection of ring 558, other removable connecting mechanisms may be used, including snap connections, bayonet connections and the like.

The entire length of outer sheath 500 and valve cover 550 may be covered by a flexible, elastic, fluid-impermeable jacket 560 to seal the slits in the proximal portion 506 and the coil/braid portion 514/516 in the flexible portion 512 of the outer sheath and the cuts 552 and slits 554 in the valve cover. Alternatively, jacket 560 may extend only from valve cover 550 to a location just proximal of coil/braid portion 514/516, and another fluid-impermeable jacket may be applied over the slits in the proximal portion 506 of outer sheath 500. In yet another arrangement, jacket 560 may be interposed between the coiled layer 514 and braided sleeve 516 of outer sheath 500, with other fluid-impermeable layers over valve cover 550 and the slits in the proximal portion 506 of the outer sheath. Any of these arrangements may be employed, as long as a liquid-tight structure results. The purpose of the fluid-impermeable layer or layers is to enable the entirety of catheter assembly 400 to be flushed with a saline solution to remove all air so that no air emboli are introduced into the patient's body during use of delivery system 200. Jacket 560 may be formed from a tube of Texin® synthetic resin available from Covestro LLC, or from another elastic material. After the application of jacket 560, the proximal and distal ends of the jacket may be bonded to the underlying structure using thermal adhesives, UV-bonded adhesives, other types of adhesives, thermal boding, heat shrinking or other techniques. Subsequently, jacket 560 may be cleaned and its surface activated by a plasma treatment process and a hydrophilic coating may be applied thercover. The cleaning and surface activation process helps to ensure that the hydrophilic coating adheres to the surface of layer 560.

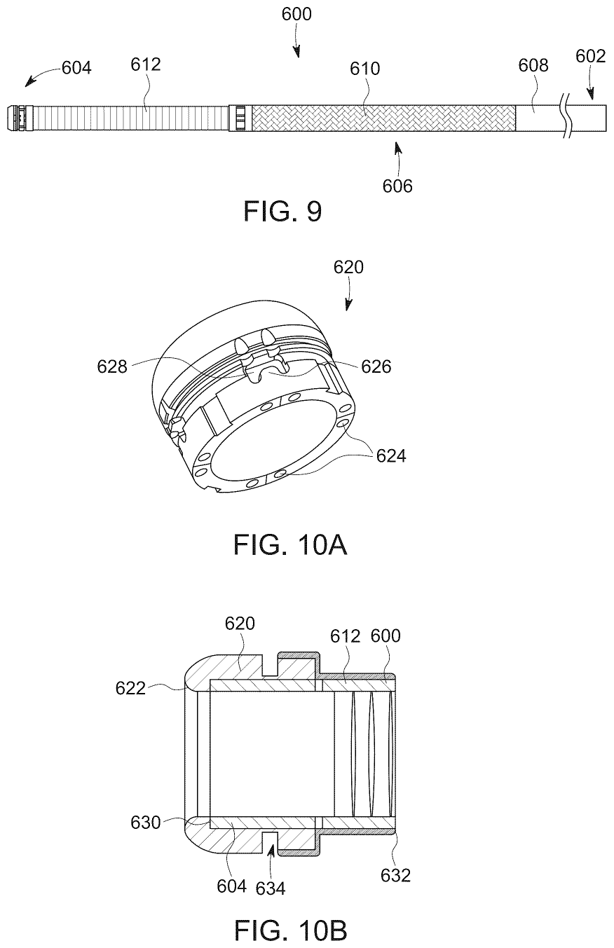

Steering catheter 600 may be nested within outer sheath 500 and may extend from a proximal end 602 connected to steering catheter handle 132 to a distal end 604. Steering catheter 600 is configured to be selectively curved to facilitate navigation through the patient's vasculature and portions of the heart. Referring to FIG. 9, steering catheter 600 may be formed from a stainless steel hypotube 606 having a proximal section 608, an intermediate section 610 and a distal section 612. The proximal section 608 of hypotube 606 may be uncut to provide steering catheter 600 with a desired amount of stiffness, torqueability and pushability, as well as the ability to form a leakproof connection with steering catheter handle 132. Intermediate section 610 and distal section 612, on the other hand, may be laser cut in a way that enables each section to have sufficient flexibility to achieve a desired bending radius. Intermediate section 610 may be laser cut in an interrupted spiral pattern along its length, much like the proximal portion 506 of outer sheath 500, and may include an outer metal braided layer. Distal section 612 may include a plurality of V-shaped or island cuts and slits, similar to those in valve cover 550, formed along opposite sides of hypotube 606, with each successive pair of cuts and slits being offset in the circumferential direction from an adjacent pair of cuts and slits by 90°. This arrangement of cuts and slits enables distal section 612 to be deflected within one, two or three planes orthogonal to one another. Each of the cuts and slits end in a further cut forming an L shape or a T shape to avoid stress concentration at the ends of the laser cuts. Although the island cuts are described as V-shaped (or triangular), they may have one or more other shapes, such as diamond, square, rhombohedral, rectangular, circular, oblong, other elliptical, other polygonal, irregular, or combinations thereof.

A steering or tip ring 620, shown in FIGS. 10A and 10B, may be secured to the distal end 604 of steering catheter 600. To minimize interactions between steering catheter 600 and extension catheter 700, described below, it is beneficial to omit sharp edges at the distal end of the steering catheter, especially on the inner surfaces at or near the distal end of the steering catheter. As best shown in the cross-sectional view of FIG. 10B, for example, tip ring 620 may have an angled or rounded distal edge 622 that enables steering catheter 600 to more effectively move and slide within outer sheath 500 without binding, and that prevents binding against components intended to translate within the steering catheter, such as extension catheter 700.

To selectively control the curvature of the distal section 612 of steering catheter 600, the steering catheter may be provided with a plurality of tension cables (not shown). The tension cables may travel from steering catheter handle 132 through a plurality of polymer tubes, such as those formed from polytetrafluoroethylene (PTFE), a polyimide, or nylon, to the tip ring 620 at the distal end 604 of steering catheter 600. In one embodiment, steering catheter 600 may include four such tubes equally spaced at 90° intervals around the circumference of hypotube 606. In another embodiment, steering catheter 600 may include four pairs of such tubes (a total of eight tubes), with the pairs of tubes equally spaced at 90° intervals around the circumference of hypotube 606. Any number of these tubes may be provided depending on the various directions of deflection that may be desired. Tip ring 620 may include a plurality of apertures 624 spaced in pairs around its circumference, with a seat 626 and a cutout 628 longitudinally aligned distally of each aperture pair. Each tension cable may travel distally through one of the tubes, through one of a pair of apertures 624 in tip ring 620, through a cutout 628 and around a seat 626, and then proximally though the other aperture in the pair. At that point, the tension cable may travel proximally through the same tube (when there are four tubes) or through another tube (when there are eight tubes) back to the proximal end 602 of steering catheter 600 where the cable ends are attached to and controlled by steering catheter handle 132, as will be explained below. Seat 626 limits the radius of curvature of the tension cable as it turns 180°, thereby reducing the risk of pinching and the impact of stresses that may damage the tension cable. The routing of the tension cables through tip ring 620 also provides a secure attachment without the need to rely on welding or adhesives to make that attachment. A polymer layer 632 formed, for example, from Pebax, may be provided as an outer layer surrounding a portion of tip ring 620 and steering catheter 600, including the tubes guiding the tension cables. Layer 632 may be melted to reflow the polymer over the tension cable tubes, tip ring 620 and steering catheter 600. A shrink tube (not shown) formed from PTFE, fluorinated ethylene propylene or another polymer may be applied over layer 632 so that, as layer 632 melts, the shrink tube will shrink, applying radial compression to layer 632 so that it tightly conforms to the underlying structures, holding the tension cable tubes tightly against steering catheter 600. Once the reflow process has been completed, the shrink tube may be removed. A circumferential slot 634 in tip ring 620 may facilitate forming a clean cut at the distal end of polymer layer 632 and provides a recess for receiving any excess of the melted polymer to avoid any loose or extraneous material at the distal end of steering catheter 600.

As noted above, the distal section 612 of steering catheter 600 may include a series off cuts and slits that enable the distal section to be deflected in two planes orthogonal to one another. One set of tension cables may be manipulated to deflect distal section 612 to a desired angle in a first plane. Another set of tension cables may be manipulated to deflect distal section 612 to a desired angle in a second plane orthogonal to the first plane. However, if both sets of tension cables are connected to tip ring 620 at the distal end 604 of steering catheter 600, deflecting distal section 612 in the second plane may alter the angle to which the distal section had been deflected in the first plane. To alleviate this problem, one set of tension cables may be connected to tip ring 620 at the distal end 604 of steering catheter 600, and another set of tension cables may be connected to a ring (not shown) spaced in distal section 612 proximally of the tip ring. As a result, distal section 612 may be deflected in two planes independently of one another, with the deflection in one plane not impacting the deflection in the other plane. Further, one or more tension cables may additionally or alternatively be coupled at its distal end to the intermediate section 610 of steering catheter 600 to provide the ability to selectively control the curvature of the intermediate section.

Since the tension cables essentially transmit only pull force, tip ring 620 will be pulled proximally to one side during steering manipulations. This can put a lot of stress on the joint between tip ring 620 and the distal portion 612 of steering catheter 600. To provide a stable joint sufficient to withstand these stresses, tip ring 620 may include a step 630 that forms an inner diameter on the proximal end of the ring that is substantially similar to the diameter on the distal end 604 of steering catheter 600. Steering catheter 600 may be inserted into tip ring 620 such that step 630 bears against the distal end 604 of the steering catheter, thereby providing support as the tip ring is pulled proximally.

With the distal end 604 of steering catheter 600 inserted fully into tip ring 620 and abutting step 630, the tip ring may be laser welded to the steering catheter with a laser seam weld. The laser weld may be a continuous line where the inner diameter at the proximal end of tip ring 620 contacts the outer surface of steering catheter 600. A continuous seam weld may beneficially smooth out potential tolerance mismatches between the inner diameter of tip ring 620 and the outer diameter at the distal end 604 of steering catheter 600.

One of the functions of delivery system 200 is to position the distal tip of catheter assembly 400 so that prosthetic mitral valve 100 can be deployed in the proper location. This may be accomplished by bending steering catheter 600 in two separate planes via a transseptal approach.

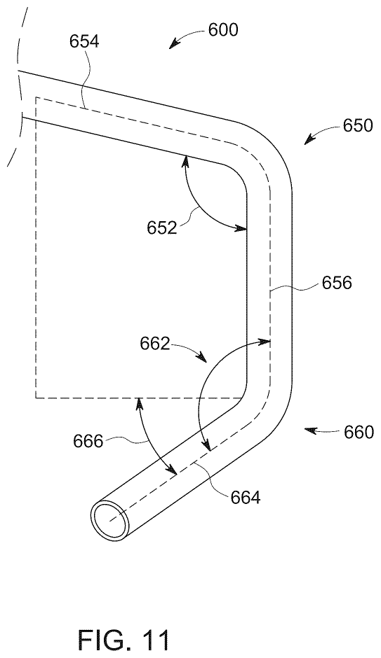

FIG. 11 illustrates an example of a series of complex bends that the distal section 612 of steering catheter 600 may make during the delivery, recapture or repositioning of a prosthetic mitral valve. While accessing the mitral valve annulus, the distal section 612 of steering catheter 600 may be steered in at least two planes of motion that may be substantially perpendicular to one another. In the illustrated example, steering catheter 600 has a first bend 650 with a first bend angle 652 measured between the longitudinal axis 654 of a first portion of the steering catheter and the longitudinal axis 656 of a second portion of the steering catheter. In some embodiments, the first bend angle 652 may be between about 40° and about 150°, more often between about 90° and about 120°, or about 105°. Steering catheter 600 may also have a second bend 660 with a second bend angle 662 between longitudinal axis 656 and the longitudinal axis 664 of a third portion of the steering catheter. In one embodiment, the second bend angle 662 may be between about 45° and about 135°, or about 60°. The second bend 660 may also have a rotational angle 666 relative to the plane in which the first longitudinal axis 654 and the second longitudinal axis 656 line. In other words, the rotational angle 666 reflects the amount of rotation of the third longitudinal axis 664 relative to the direction of the first bend 650. In some embodiments, the rotational angle 666 may be between about 45° and about 135°, or about 60°. In some embodiments, steering catheter 600 may achieve bend angles of up to 180°.

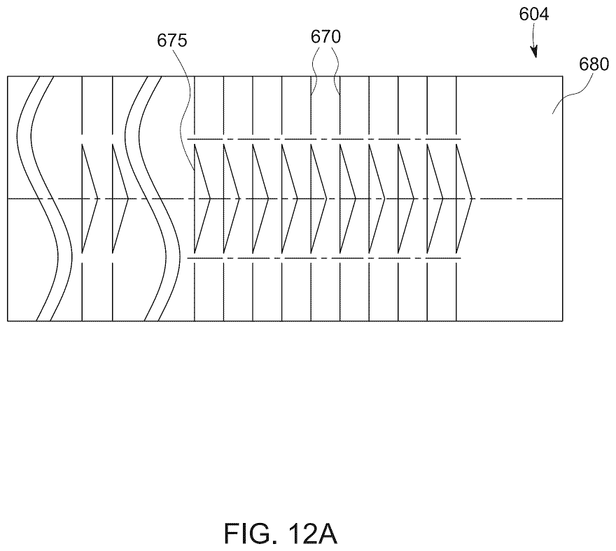

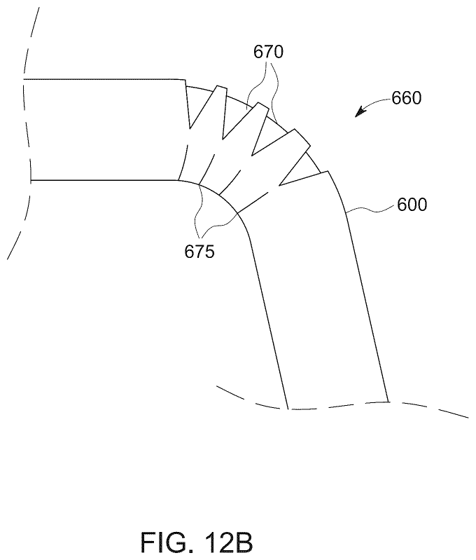

Although steering catheter 600 has been described above with interrupted spiral cuts in intermediate section 610 and V-shaped or island cuts and slits in distal section 612, other cutting patterns are possible. Various cutting patterns can be used in different sections of steering catheter 600 to produce the desired bends. For example, referring to FIG. 12A, each section can include cut patterns that may include one or more slits 670 and/or one or more island cuts 675. Slits 670 may enable longitudinal forces to be transmitted along steering catheter 600 and also allow expansion of the slits when the steering catheter is deflected in a direction opposite the slits. Island cuts 675 may collapse when the steering catheter is deflected in the direction of the island cuts. For example, slits 670 and island cuts 675, when located on opposite sides of steering catheter 600, may direct preferential bending of the catheter, as shown by exemplary bend 660 in FIG. 12B. Island cuts 675 may be formed so that they progressively change in size in the length direction of steering catheter 600. For example, island cuts 675 may progressively get smaller in the distal direction along the length of steering catheter 600. Such a cut pattern would provide a gradient bend that gradually increases in deflection at successively more proximal sections.

The distal-most section of steering catheter 600 preferably has a relatively straight section. Referring to FIG. 12A, this may be manifest as an uncut section 680 at the distal end 604 of steering catheter 600. The uncut, relatively straight section 680 allows the components advancing past the distal end 604 of steering catheter 600 to continue along a straight path. For example, by pointing the distal end 604 of steering catheter 600 directly at the mitral valve annulus, the components advancing distally beyond the steering catheter (such as, for example, extension catheter 700) will continue on a straight trajectory toward/through the annulus. Moving proximally from straight, uncut section 680, the bend in steering catheter 600 forms gradually before increasing to form the full bend. Other methods of achieving a straight section at the end of steering catheter 600 are also possible. In one example, can 720 described below can be attached directly to the tip ring 620 of the steering catheter, and the can may be provided with a straight section. In another example, the tip ring 620 of the steering catheter itself may be provided with a straight section.

To provide effective steering and positioning at the mitral annulus, the distal section 612 of steering catheter 600 may be cut with a pattern that enables a bending radius of about 15 mm or less, or between about 5 mm and about 15 mm. The intermediate section 610 of steering catheter 600 may be cut to enable a bending radius of between about 30 cm and about 45 cm. The proximal section 608 of steering catheter 600 may remain uncut to ensure that the steering catheter has sufficient stiffness, torque-ability and push-ability to effectively operate, and to enable a leakproof connection between the steering catheter and steering catheter handle 132.

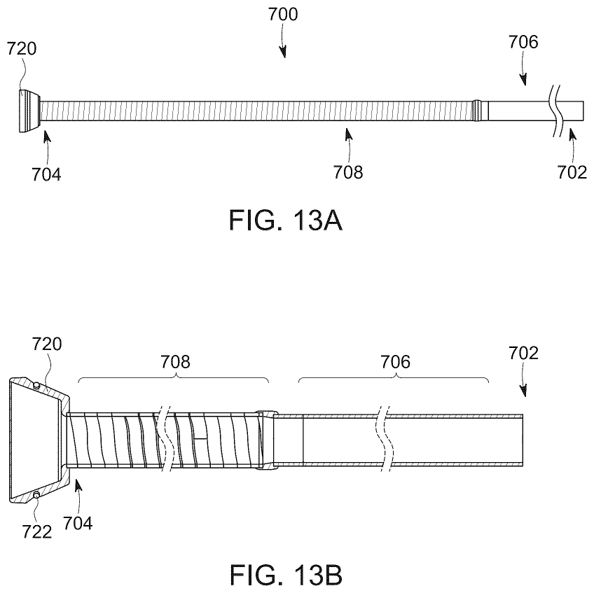

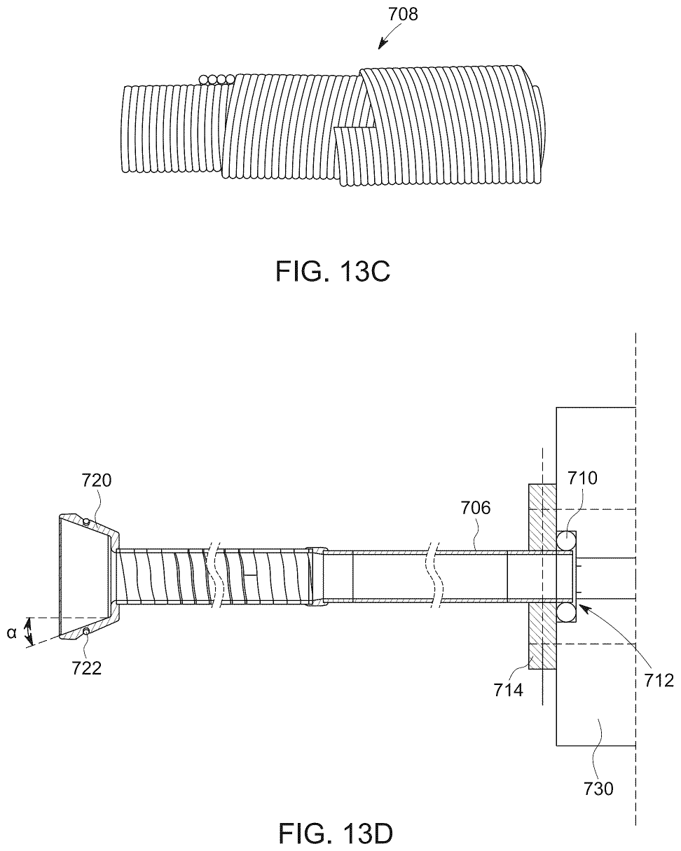

Nested within steering catheter 600 is an extension catheter 700, one embodiment of which is shown in FIGS. 13A-13C. Alternate embodiments of extension catheter 600 having a structure and features other than those described herein may also be used. Extension catheter 700 may have to withstand relatively high compression forces during the deployment of a prosthetic heart valve. For example, during the release of prosthetic mitral valve 100 by the retraction of outer sheath 500, the countervailing compression force on extension catheter 700 may be on the order of about 30 to 50 lbs. Extension catheter 700 must also have sufficient flexibility to allow for proper deflection and curvature to achieve a desired position in the mitral valve annulus. A coil structure, such as in a distal section of extension catheter 700, may be beneficial because it has high flexibility and is also able to withstand high compression forces. In some embodiments, the coil may be a flat wire stacked coil (i.e., a coil in which adjacent turns contact one another with no gaps between them), which has been found to provide an effective balance between flexibility and compressive strength. The stacked coil portion of extension catheter 700 optionally may be covered by a braided tube (not shown, but similar in construction to the coil/braid portion 514/516 of outer sheath 500) to minimize interference with the distal end of steering catheter 600 as the turns of the stacked coil separate from one another when forced into a tight bend.

Referring to FIGS. 13A and 13B, extension catheter 700 extends from a proximal end 702 to a distal end 704, with a proximal section 706 adjacent the proximal end and a distal section 708 adjacent the distal end. The proximal section 706 of extension catheter 700 may be formed from a stainless steel hypotube having a laser cut interrupted spiral pattern of slits (not shown) beginning at a spaced distance from proximal end 702. In one embodiment, the proximal end 702 of the hypotube may be fitted with an O-ring 710 and may be positioned within a circular recess 712 in extension catheter holder 730, as shown in FIG. 13D. A C-clamp 714 may be assembled around the hypotube and connected to extension catheter holder 730 with a plurality of screws, compressing O-ring 710 to form a fluid-tight seal. However, other mechanisms for connecting extension catheter 700 to extension catheter holder 730 with a fluid-tight seal are also contemplated herein. The distal section 708 of extension catheter 700 may be formed with a tri-coil structure, rather than the stacked coil structure described above. That is, distal section 708 may be formed with three layers of wire coils, with the inner and outer layers being wound in the same direction, and the middle layer being wound in a direction opposite that of the inner and outer layers. An illustration of the tri-coil structure is shown in FIG. 13C. In one example, the inner coil may consist of a single wire or filar wound into a coil; and the middle and outer layers may each consist of a bundle of six filars arranged side-by-side and wound into a coil. Each of the individual filars may have a rectangular cross-section with a thickness of about 0.004 in. and a width of about 0.012 in. The foregoing tri-coil structure is merely exemplary, as the individual coil layers may have bundles with a greater or lesser number of filars (1×5×5, 1×7×7, 1×8×8, 1×7×8, etc.), and the filars may have different dimensions in cross-section. It is also possible to leave a gap the size of one filar or more between filar bundles. The three coils are coextensive and are welded to one another at their ends, as well as welded to proximal section 706. The tri-coil structure provides distal section 708 with enough flexibility to enable extension catheter 700 to be advanced distally through catheter assembly 400 when the distal end of steering catheter 600 has been deflected into a tight curve to align prosthetic mitral valve 100 with mitral valve annulus 158. As a result, extension catheter 700 is better able to maintain the position of prosthetic mitral valve 100 relative to native mitral annulus 158 as catheter assembly 400 is manipulated to deploy the prosthetic valve.

Extension catheter 700 includes a can structure 720 that may be laser welded to distal end 704. Can 720 is configured to constrain and hold at least the atrial petals 114 of prosthetic mitral valve 100. Without such constraint, atrial petals 114 might bend outwardly under compression when valve cover 550 is retracted, locking the prosthetic mitral valve inside the valve cover and making it more difficult to unsheathe or re-sheathe it.

Can 720 may be sufficiently long to aid in maintaining coaxial alignment of the distal end 704 of extension catheter 700 with catheter assembly 400 to avoid or minimize unwanted tilting, but sufficiently short to not interfere with the release of atrial petals 114 at the proper position relative to mitral valve annulus 158. Can 720 also provides an effective structural surface to act as a counterforce to maintain the prosthetic heart valve in a proper pre-deployed position when outer sheath 500 is retracted. In that regard, the atrial petals 114 of prosthetic mitral valve 100 will taper inward toward the centerline of the prosthetic valve when the valve is in a collapsed condition, forming an angle a with the longitudinal axis of extension catheter 700. The inner sidewall 724 of can 720 preferably also forms an angle of about a with the longitudinal axis of extension catheter 700 so that the radial expansion force exerted by atrial petals 114 is applied more uniformly to can 720 and the atrial petals are held more securely in the can. Additionally, the depth of can 720 should be sufficient for atrial petals 114 to nest in the can and be held securely in place, but not so deep that prosthetic mitral valve 100 has to move too great a distance to be released from the can. For certain prosthetic mitral valves, the depth of can 720 may be about 1-15 mm, or about 2-10 mm, or about 2-7 mm, or about 3-4 mm. In some embodiments, one or more edge portions of can 720 may include a taper and/or smooth surface for easier sliding of the can within outer sheath 500. Can 720 may be formed from a sufficiently dense metal, such as stainless steel, that it can be seen under x-rays. Optionally, an outer groove around the circumference of can 720 may include a wire 722 formed from tantalum or other highly radiopaque material to identify the end of extension catheter 700 under x-rays, even while the can is within valve cover 550. It is important to be able to identify when can 720 is longitudinally aligned with the open end of valve cover 550 as this indicates when full deployment of ventricular anchor 108 has been achieved. Rather than radiopaque wire 722, any other radiopaque marker may be applied to can 720 for this purpose.

A suture catheter 800 may be positioned within extension catheter 700. Generally, suture catheter 800 must have a sufficiently flexible distal end that is able to accommodate the tight curves at the distal end of steering catheter 600. In addition, suture catheter 800 must withstand substantial tension during the loading of prosthetic mitral valve 100 into valve cover 550 and must maintain axial tension on the prosthetic mitral valve prior to deployment. By maintaining such axial tension, suture catheter 800 may aid in maintaining the atrial petals 114 of the prosthetic valve within can 720 and the position of the prosthetic valve within catheter assembly 400, as will be described further below.

As will be explained, a nosecone catheter 900 may be nested within the lumen of suture catheter 800. It is beneficial for each of the components of catheter assembly 400 to be able to slide relative to one another under various challenging curvatures to which the catheter assembly is subjected during a transseptal approach to the mitral valve annulus. Accordingly, it is beneficial for nosecone catheter 900 to be able to translate freely within suture catheter 800. Providing a smooth inner surface within suture catheter 800 to facilitate the translation of nosecone catheter 900 therein, however, can be challenging. In preferred embodiments in which suture catheter 800 comprises a laser cut hypotube, it can be difficult to achieve a smooth inner surface. For example, the laser cut hypotube may be too small/tight to enable sufficient electro-polishing. While honing is another option, the length of the laser cut portion of the hypotube makes such procedure difficult. Other techniques, such as extrude honing, may not produce the desired smoothness on the inner lumen of steering catheter 800.

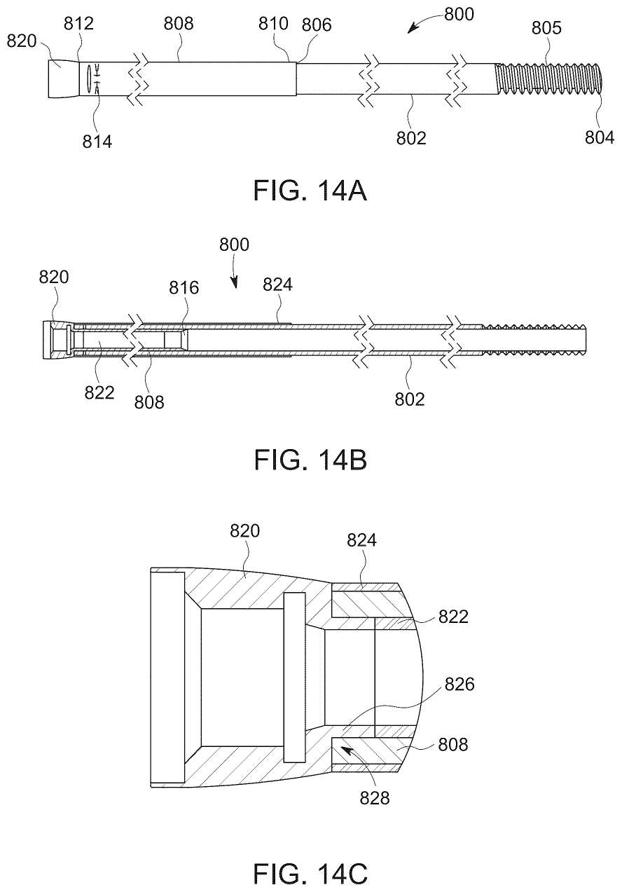

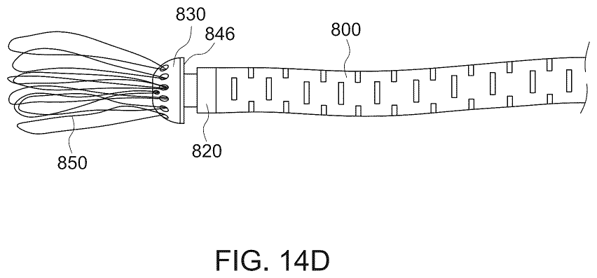

One embodiment of a suture catheter 800 having smooth inner walls is shown in FIGS. 14A-C, although embodiments of the suture catheter having alternative features and constructions are contemplated herein. Suture catheter 800 has a relatively long proximal portion 802 that may be formed from a stainless steel hypotube having a proximal end 804 and a distal end 806. A portion of the hypotube adjacent proximal end 804 may include external threads 805 for attachment to the internal threads of a suture catheter control 870, as described below, although other mechanisms for attaching the suture catheter to the suture catheter control may also be used, including a snap fit connector, retaining clip, bayonet connector, and the like. The proximal portion 802 of suture catheter 800 may include an interrupted spiral pattern of slits cut along its length, the slits terminating at a spaced distance from external threads 805. Suture catheter 800 may also have a relatively short distal portion 808 formed from a length of hypotube having a proximal end 810, a distal end 812 and a pattern of dog bone slits 814 laser cut along its length. The dog bone slits formed in the distal portion 808 of suture catheter 800 enable the distal portion to form tight bends in different planes while still withstanding a high degree of tension. A thin-walled stainless steel ring 816 may be laser welded at one end to the proximal end 810 of the distal portion 808 of suture catheter 800 so that it protrudes therefrom. The distal end 806 of the proximal portion 802 of the suture catheter may then be assembled over the protruding portion of ring 816, which aligns proximal portion 802 with distal portion 808 so they can be joined together by a scam weld.

A proximal suture ring 820 may be laser welded to the distal end 812 of the distal portion 808 of suture catheter 800. Proximal suture ring 820 may have a tubular proximal portion 826 sized to fit within the distal end 812 of distal portion 808. Proximal portion 826 may define a step 828 with the remainder of proximal suture ring 820, the step being sized so that the distal portion 808 of suture catheter 800 mates smoothly with the external surface of the proximal suture ring and does not leave any exposed sharp edges. Proximal suture ring 820 may be internally threaded at its distal end to mate with the external threads of a distal suture ring 830, shown in FIG. 14D. A plurality of tethers 850 connected to distal suture ring 830 may releasably connect to attachment features on prosthetic mitral valve 100. As a result, as suture catheter 800 is retracted relative to outer sheath 500, tension will be applied through distal suture ring 830 and tethers 850 to the prosthetic mitral valve, drawing it into the can 720 of extension catheter 700 and valve cover 550.

A free-floating polytetrafluoroethylene tube 822 may line the lumen of distal portion 808 from proximal suture ring 820 to ring 816, covering any sharp edges and weld lines resulting from the assembly of the components and providing a low-friction surface to facilitate the sliding of nosecone catheter 900 within suture catheter 800. Tube 822 is captured and held in place between proximal suture ring 820 and ring 816. In addition, a low friction tube 824 may be heat shrunk around the outside of distal portion 808 and the spiral cut section of proximal portion 802 to reduce the friction between the outside diameter of suture catheter 800 and the inside diameter of extension catheter 700. Tube 824 may be formed from polytetrafluoroethylene or, more preferably, from a fluorinated ethylene propylene, although other low friction materials may be used. An additional stainless-steel ring (not shown) may be laser welded around the outside of distal portion 808 near its proximal end 810. When tube 824 is heat shrunk around suture catheter 800, the outer ring may help prevent the tube from sliding along the length of the suture catheter as it translates within catheter assembly 400. The use of fluorinated ethylene propylene for tube 824 enables the tube to bend without forming wrinkles that could impede the ability of steering catheter 800 to slide relative to other components of catheter assembly 400.

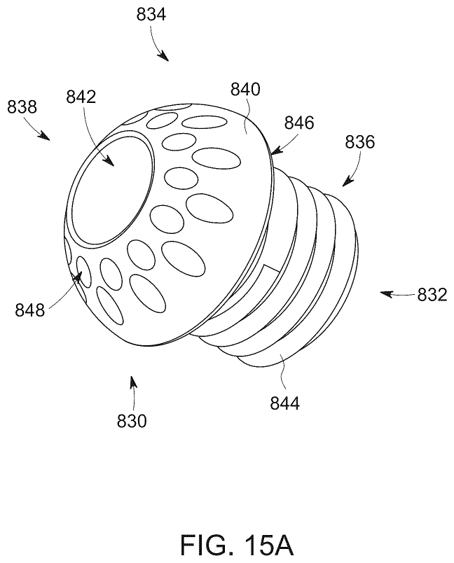

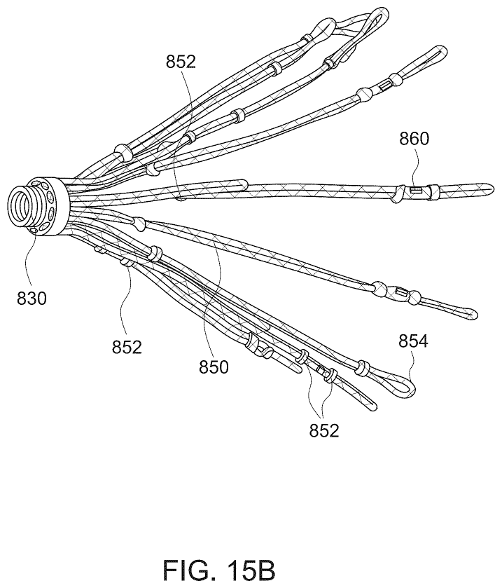

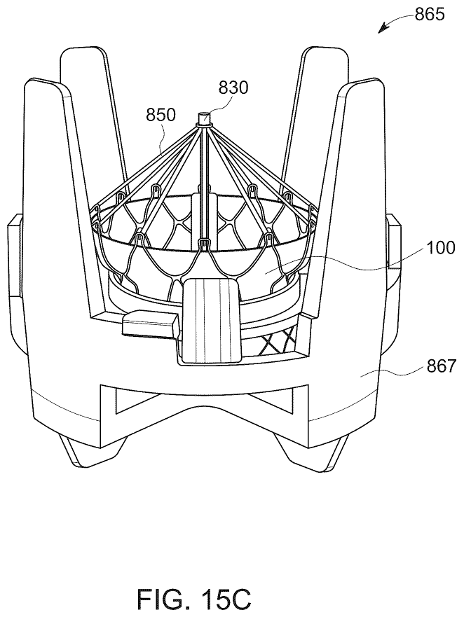

Distal suture ring 830 is connectable to the proximal suture ring 820 of suture catheter 800. A preferred distal suture ring 830 for use with catheter assembly 400 is shown in FIG. 15A and described in U.S. Provisional Application No. 63/228,269, the disclosure of which is hereby incorporated by reference herein. However, the present disclosure contemplates the use of alternative embodiments of the distal suture ring that may have a different construction and different features. Distal suture ring 830 extends in a longitudinal direction between a proximal end 832 and a distal end 834 and has somewhat of a mushroom shape with a generally cylindrical body 836 at the proximal end terminating in an enlarged head 838 with a domed or hemispherical surface 840 at the distal end. A lumen 842 may extend in the longitudinal direction through the cylindrical body 836 and head 838 of distal suture ring 830 and may be sized to receive guidewire 975 and nosecone catheter 900 therethrough. As illustrated, cylindrical body 836 may be formed with external threads 844 sized and shaped to securely connect to the internal threads in the proximal suture ring 820 of suture catheter 800. However, other mechanisms for fastening distal suture ring 830 to suture catheter 800 are also contemplated so long as they are sufficiently strong to withstand the substantial tensile forces that will be exerted thereon as prosthetic mitral valve 100 is collapsed and loaded into valve cover 550. Since the proximal suture ring/distal suture ring assembly is not bendable, it is preferable that the assembly be as short as possible in the longitudinal direction of suture catheter 800.

The head 838 of distal suture ring 830 has a diameter that is substantially larger than the diameter of cylindrical body 836, thereby defining a shoulder 846 extending around the cylindrical body and facing toward the proximal end 832 of the suture ring. A plurality of round apertures or bores 848 may extend through head 838 from shoulder 846 to surface 840. Bores 848 may extend parallel to one another and parallel to the longitudinal direction of distal suture ring 830, and each has a diameter sized to receive a length of suture thread. One or more suture threads may be attached to the head 838 of distal suture ring 830. The suture threads may be formed of various materials, either man-made or natural, or a combination thereof. Examples of natural suture materials may include, but are not limited to, silk, linen, and catgut. Examples of synthetic suture materials may include, but are not limited to, textiles such as nylon or polyester, or flexible metallic cables. Referring to FIG. 15B, an elongated suture thread may be threaded through a plurality of the bores 848 in distal suture ring 830 to form tethers 850. A plurality of knots 852 may be formed in tethers 850 to secure the tethers to distal suture ring 830. Knots 852 may also prevent adjacent lengths of suture thread from separating too far from one another to create a large loop or lasso that may potentially become entangled with prosthetic mitral valve 100 as it is being deployed. Knots 852 may also form a closed attachment loop 854 at the free ends of tethers 850. Attachment loops 854 are intended to releasably hook onto the pins 118 of prosthetic mitral valve 100 and to apply tension to assist in collapsing the prosthetic mitral valve during loading into valve cover 550, as described more fully below. A radiopaque marker 860 may be provided on each of tethers 850 and may be captured between a pair of knots 852. Radiopaque markers 860 help visualize the locations of the tethers, and in particular the positions of attachment loops 854, during the deployment of prosthetic mitral valve 100 in a patient. A prosthetic mitral valve assembly 865 is shown in FIG. 15C and consists of distal suture ring 830 attached to prosthetic mitral valve 100 by a plurality of tethers 850, with a portion of a packaging assembly 867 securing the prosthetic mitral valve and distal suture ring so that the tethers are tensioned. Details of prosthetic mitral valve assembly 865 are shown and described in U.S. patent application Ser. No. 17/317,377, filed May 11, 2021, the disclosure of which is hereby incorporated by reference herein.

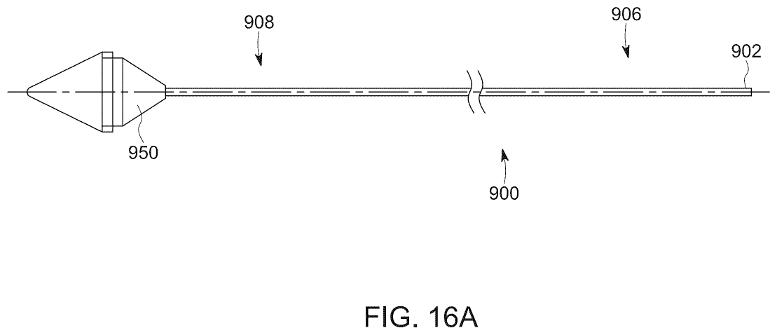

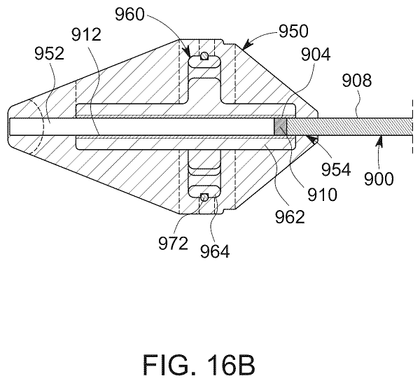

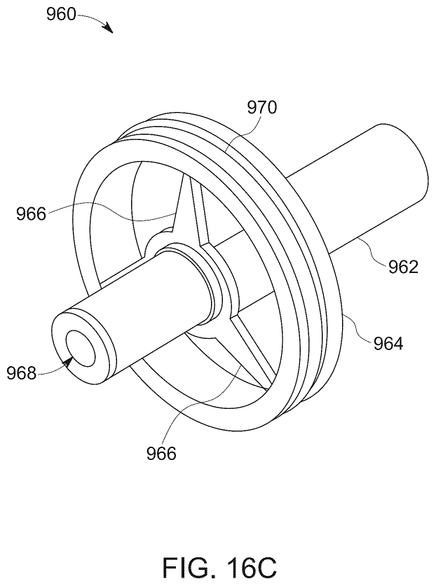

Nosecone catheter 900, an embodiment of which is illustrated in FIG. 16A, may be positioned within the lumen of suture catheter 800 and may comprise the innermost component of catheter assembly 400. However, the use of alternative embodiments of the nosecone catheter that may have a different construction and different features is contemplated herein. Nosecone catheter 900 has a lumen therethrough configured to receive guidewire 975. For example, the lumen of nosecone catheter 900 may have a diameter of about 0.037 inches so as to be compatible with a standard 0.035-inch guidewire, although other sizes may be utilized according to particular application needs. Nosecone catheter 900 extends from a proximal end 902 to a distal end 904, shown in FIG. 16B. A proximal portion 906 of nosecone catheter 900 may be formed from a very thin-walled stainless steel hypotube that, because of its thin wall and small diameter, is flexible. As a result, laser cuts along the length of the hypotube to increase its flexibility are not necessary. However, nosecone catheter 900 may also be formed from a polymer, such as polyimide, nylon, etc. Adjacent distal end 904, nosecone catheter 900 may have a distal portion 908 that includes 2-3 concentric layers of coiled wire laser welded to the hypotube of proximal portion 906. The coiled structure of distal portion 908 provides much greater flexibility than that of proximal portion 906, enabling nosecone catheter 900 to follow the tight bends of steering catheter 600 within the restricted confines of the heart. The innermost coil of distal portion 908 may be wound in a first direction and the overlying coil may be wound in the opposite direction. In addition to providing improved flexibility, the coiled structure of distal portion 908 provides good resistance to any tension forces applied to nosecone catheter 900, thereby minimizing the possibility of the nosecone catheter breaking under such forces. To further improve the strength of distal portion 908 under tension, a third coil layer may be applied over the first two coiled layers, with the third coil layer being wound in the direction opposite that of the underlying coil layer (i.e., in the same direction as the innermost coil).

In order to be able to load prosthetic mitral valve 100 into a fully assembled catheter assembly 400, it is preferable to be able to remove nosecone 950 from nosecone catheter 900. This will enable the distal end 904 of nosecone catheter 900 to be inserted through the prosthetic mitral valve 100 during the loading procedure, rather than having to slide the prosthetic mitral valve over the entire length of the nosecone catheter from its proximal end 902. In one embodiment, the distal portion 908 of nosecone catheter 900 may include a stainless-steel tube adapter 910 (FIG. 16B) laser welded to the free ends of the coils. Tube adapter 910 may be externally threaded at its distal end for detachably mating with internal threads in nosecone 950, as described below. (Where nosecone catheter 900 is formed from a polymer, a stainless-steel adapter having external threads may be glued to the distal end of the nosecone catheter. However, alternative mechanisms for removably attaching nosecone 950 to nosecone catheter 900 may also be used, including a snap fit connection, a bayonet connection or other known releasable connections.

Embodiments of nosecone 950 that may be used with delivery system 200 are described in detail in U.S. Patent Publication No. 2020/0323634, the disclosure of which is hereby incorporated by reference herein. One such embodiment of nosecone 950 that is connectable to nosecone catheter 900 is illustrated in cross-section in FIG. 16B. Nosecone 950 provides an angled, atraumatic shape which assists in advancing catheter assembly 400 through the patient's vasculature and inter-atrial septum 154 to mitral valve annulus 158, all while minimizing damage to the vasculature and cardiac tissue. Nosecone 950 may be injection molded from a relatively soft polymeric material, such as polyurethane, mixed with a foaming agent, such as Foamazol® available from Bergen International LLC. The foaming agent creates small voids within nosecone 950 which enable the nosecone to be visible under ultrasound imaging. The voids also reduce the mass of nosecone 950, facilitating the injection molding process. In an alternate embodiment, nosecone 950 may be formed from a different polymer, such as silicon. The polymeric material used to form nosecone 950 may also incorporate a radiopaque material, such as barium sulfate, whereby the nosecone produces a ghost-like image that is visible under x-rays. Following injection molding, nosecone 950 may be coated with a hydrophilic coating.

Nosecone 950 may be molded around a rigid polymer insert 960, an embodiment of which is shown in FIG. 16C. Inserts of the type that may be incorporated in nosecone 950 are shown and described in U.S. Patent Publication No. 2019/089627, the disclosure of which is hereby incorporated by reference herein. Insert 960 may be completely encased by the softer polymer forming nosecone 950 and may include an elongated shaft 962 surrounded coaxially by a rim 964. Rim 964 may be connected to shaft 962 by a plurality of spokes 966 that space the rim from the shaft and orient the rim in a plane that is perpendicular to the longitudinal axis of the shaft. Shaft 962 may have a lumen 968 extending through the entire length thereof in alignment with a first bore 952 in the distal end of nosecone 950 and a second bore 954 in the proximal end of the nosecone. The diameters of lumen 968 and bores 952 and 954 are such that guidewire 975 can be inserted in sliding relationship therethrough. At its proximal end, shaft 962 may include internal threads (not shown) sized to mate with the threads on tube adapter 910 to assemble nosecone 950 to nosecone catheter 900. In that regard, bore 954 at the proximal end of nosecone 950 has a diameter sized to receive tube adapter 910 and the distal portion 908 of nosecone catheter 900 therein. Lumen 968 and bores 952 and 954 may be lined with a Pebax® tube 912 (trademark of Arkema France) that extends through the entire length of nosecone 950, with the exception of the threaded portion at the proximal end of shaft 962, which must be kept clear for threaded engagement with the threads on tube adapter 910. At the distal end of nosecone 950, tube 912 prevents guidewire 975 from cutting through the soft polymer forming the nosecone when the guidewire is bent. At the proximal end of nosecone 950, tube 912 prevents the softer polymer material from sticking to a mold pin forming bore 954 during the molding of the nosecone. Rim 964 may include a groove 970 along its outer circumference. Groove 970 is adapted to receive a radiopaque and/or echogenic material, such as a tantalum wire 972, so that the position and orientation of rim 964 is clearly visible under x-rays and/or ultrasound.

As noted, nosecone catheter 900 and nosecone 950 have lumens or bores therein that are configured to slidably receive guidewire 975. Guidewire 975 is a thin wire that is used to guide catheter assembly 400 from the insertion site in the patient to the mitral valve annulus 158 at which the prosthetic mitral valve is to be deployed. To enable catheter assembly 400 to track all the way to mitral valve annulus 158, it may be preferable for the distal tip of guidewire 975 to be advanced into left ventricle LV. Guidewire 975 preferably is highly flexible and kink-resistant in order to accommodate the tight turns encountered while advancing from the femoral vein and inferior vena cava 150, through septum 154 and mitral valve annulus 158, to left ventricle LV.

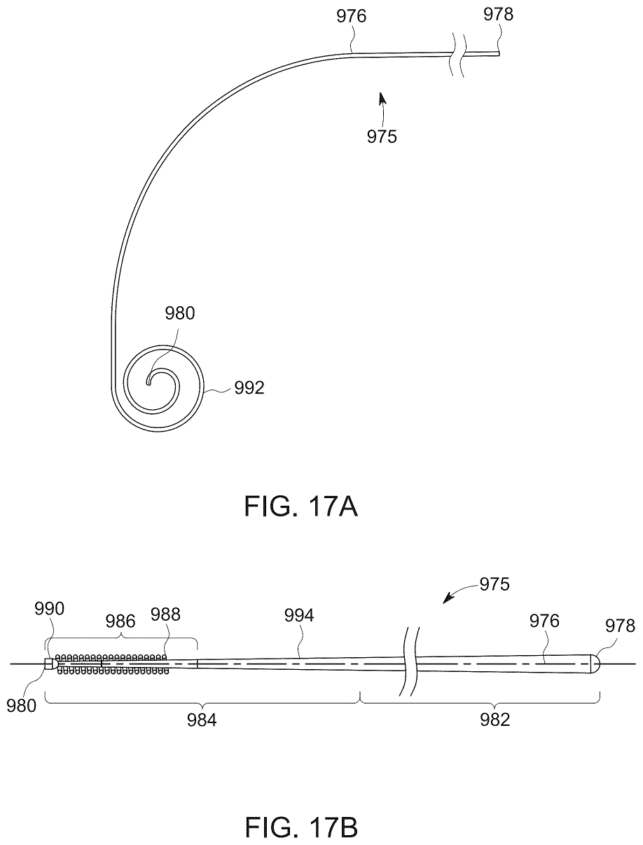

One embodiment of a guidewire 975 for use with catheter assembly 400 is shown in FIGS. 17A and 17B and described in U.S. Patent Publication No. 2020/0155803, the disclosure of which is hereby incorporated by reference herein. Guidewire 975 includes a core wire 976 that extends from a proximal end 978 to a distal end 980. Core wire 976 need not be formed from the same material along its entire length. For example, a distal portion of core wire 976 may be formed from a more flexible metal, such as nitinol, and the straighter proximal portion of the core wire may be formed from a stiffer meta, such as stainless steel. Core wire 976 has a first section 982 with a generally uniform or constant diameter closest to its proximal end 978, and a second section 984 closest to its distal end 980. Section 984 may gradually decrease or taper in diameter from section 982 to distal tip 980. A distal portion 986 of section 984 may include a coil 988 coupled to core wire 976, and the distal end 980 of the core wire may optionally include an atraumatic tip 990.

Core wire 976 may be formed of a superelastic material, such as nitinol, providing guidewire 975 with a high degree of flexibility. Coil 988 may be formed from a radiopaque metal, such as tantalum, platinum, platinum iridium, gold, silver, etc., so that the distal portion 986 of guidewire 975 is visible under x-ray imaging. A distal portion of guidewire 975, including coil 988, may have a pigtail shape, as shown in FIG. 17A. The curved configuration of the pigtail shape may prevent guidewire 975 from damaging cardiac or other tissue as it is advanced to left ventricle LV.

Guidewire 975 may be covered by an outer jacket 994. In some embodiments, the entire length of core wire 976, including coil 988, may be covered by outer jacket 994. In other embodiments, outer jacket 994 may cover the length of core wire 976 up to but not including coil 988, or may cover only the coil 988 to prevent it from being damaged. Outer jacket 994 may be formed of a polymeric material, such as a polytetrafluoroethylene tube that is applied over core wire 976 and shrunk by the application of heat. Outer jacket 994 imparts substantial lubricity and low surface friction to guidewire 975, enabling the guidewire to easily slide within nosecone catheter 900.

Guidewire 975 preferably has a length that is about twice the length of catheter assembly 400. Thus, for a catheter assembly that is about six feet (183 cm) long, guidewire 975 may have a length of about twelve feet (365 cm). In other embodiments, guidewire 975 may have a length of between about six feet (183 cm) and about fifteen feet (457 cm). The maximum diameter of guidewire 975, including outer jacket 994, may be between about 0.014 in. and about 0.035 in. For example, guidewire 975 may have a diameter of about 0.014 in., about 0.018 in. or about 0.035 in.

Although the particular nested configuration shown in FIGS. 3A and 3B and described above represents one preferred embodiment for the various components of catheter assembly 400, alternative embodiments may include a different concentric arrangement of constituent parts. For example, some embodiments may combine steering catheter 600 and outer sheath 500 into one component and/or configure the outer sheath with steering functionality, some embodiments may include more than one catheter with steering functionality, etc.

As has been described above, it is important for the user to be able to determine the position of valve cover 550 and other components of catheter assembly 400, and thus the position of prosthetic mitral valve 100, during the delivery and deployment of the prosthetic heart valve in the patient. Echo visualization using ultrasound imaging is typically employed during an insertion procedure in order to minimize the exposure of the patient to x-rays and to enable visualization of native mitral valve annulus 158 and the native mitral valve leaflets. As noted, the tantalum ring 558 on the distal end of valve cover 550 and the can 720 on the distal end of extension catheter 700 are both visible under x-ray imaging. An additional radiopaque marker, such as tantalum wire 722, optionally may be placed on can 720 to enhance its visibility under x-rays. These markers, however, are not visible under echo visualization. Rather, physicians may use certain landmarks visible under ultrasound, such as the gap between nosecone 950 and the beginning of the mitral valve or the end of the laser cuts in valve cover 550, to measure and calculate the distance from the landmarks to the mitral valve annulus to properly position the valve cover to ensure deployment of the prosthetic mitral valve at the proper position. One approach to obviate the need for the physicians to measure and calculate the proper deployment position of valve cover 550 is to place an echogenic marker on the outside of the valve cover at a position corresponding to the center of the prosthetic mitral valve held therein.

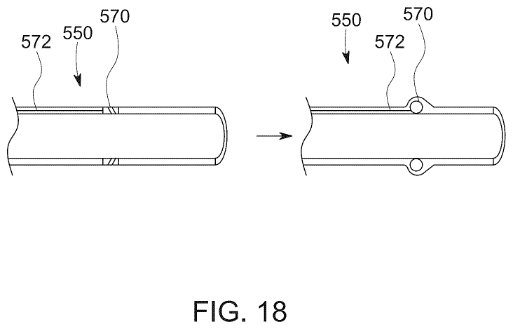

One embodiment of such an echogenic marker is shown in FIG. 18. The echogenic marker is in the form of a balloon 570 positioned between the exterior surface of valve cover 550 and the outer Texin® layer 560. Balloon 570 circumscribes an intermediate section of valve cover 550 corresponding to the approximate center of the prosthetic mitral valve therein. An inflation/deflation lumen 572 extends from balloon 570 proximally along the length of outer sheath 500 (below Texin® layer 560) to a luer or other fluid-tight fitting (not shown) at or near handle assembly 300. Inflation/deflation lumen 572 does not have a permanently open lumen therethrough. Rather, inflation/deflation lumen 572 is formed from a readily collapsible polymer film such that one side of the inflation/deflation lumen contacts the opposite side of the inflation/deflation lumen in a collapsed condition, and the inflation/deflation lumen lies flat against outer sheath 500 prior to use. When it is desired to inflate balloon 570, an inflation medium may be supplied to the balloon through the luer fitting and inflation/deflation lumen 572. Acceptable inflation media may include a gas, such as carbon dioxide or helium, or a liquid, such as saline. The movement of the inflation medium through inflation/deflation lumen 572 will cause the inflation/deflation lumen to deform to an expanded condition in which the opposite sides of the inflation/deflation lumen move away from one another, forming an open lumen to balloon 570. When balloon 570 is inflated, it will form a toroid around valve cover 550. As the Texin® layer is flexible, it will deform and not inhibit balloon inflation. The inflated balloon 570 may have a diameter of between about 0.10 mm and about 5.0 mm; between about 0.30 mm and about 3.0 mm; or between about 0.50 mm and about 2.0 mm. Since the inflated balloon 570 is devoid of structure, it is readily visible under ultrasound imaging.

As mentioned previously, catheter assembly 400 is connected at its proximal end to handle assembly 300, shown in FIG. 19, which consists of steering catheter handle 132 and a series of end rings, caps and other structures, each of which may be connected at or near the proximal end of one of the components of catheter assembly 400. The proximal end 602 of steering catheter 600 may be fixedly connected to the housing 134 of steering catheter handle 132. Steering catheter handle 132 may include one or more controls 136a, 136b to which the proximal free ends of the steering catheter tension cables are connected. Manipulation of controls 136a, 136b adjusts the tension in the tension cables to deflect or straighten the distal section 612 (and, in some embodiments, the intermediate section 610) of steering catheter 600. Manipulation of one control 136a may deflect the distal section 612 of steering catheter 600 in a first plane, and manipulation of the other control 136b may deflect the distal section of the steering catheter in a plane orthogonal to the first plane, or in a plane transverse to the first plane, depending on the pattern of laser cuts in the steering catheter. Hence, controls 136a, 136b can be adjusted in tandem to position the distal end of catheter assembly 400 and valve cover 550 at a desired position and orientation relative to mitral valve annulus 158. Although controls 136a, 136b are shown in FIG. 19 as knobs, alternative embodiments may additionally or alternatively include one or more buttons, sliders, ratcheting mechanisms, or other suitable controls capable of adjusting the tension in the steering catheter tension cables to provide steering. Illustrative structures that can be used as part of steering catheter handle 132 and/or steering catheter 600 are described in U.S. Pat. No. 7,736,388, the disclosure of which is hereby incorporated by reference herein. However, it is contemplated that mechanisms that operate in a different way and that have a different structure than steering catheter handle 132 may also be used. Since steering catheter 600 is positioned within outer sheath 500, deflection of the steering catheter causes a corresponding deflection/steering of the outer sheath. A steering catheter hemostasis valve (not shown) may be connected to the proximal end of steering catheter 600 just proximally of steering catheter handle 132, allowing for flushing of the steering catheter before use.

In the embodiment shown in FIG. 19, outer sheath 500 is assembled over steering catheter 600 and extends from its distal end 504 to its proximal end 502 which, as noted above, is fixedly connected to an outer sheath hemostasis valve 530 spaced distally of steering catheter handle 132. This positioning enables outer sheath 500 to be advanced distally relative to steering catheter 600 and retracted proximally relative to the steering catheter until hemostasis valve 530 abuts steering catheter handle 132. Although the foregoing describes steering catheter handle 132 as limiting the proximal retraction of outer sheath 500, that need not be the case, and the proximal retraction of the outer sheath can be limited by other features, for example, other components of catheter assembly 400 or features of stabilizer 1000 described below. The luer lock 532 of hemostasis valve 530 extends into and is movable along an elongated slot 142 in a rigid arm 140 fixedly connected to, and projecting distally from, steering catheter handle 132. The engagement of luer lock 532 in slot 142 also rotationally keys steering catheter 600 to outer sheath 500. As noted above, outer sheath 500 may be laser cut to provide particular preferred bending directions. However, the bending of outer sheath 500 relies on the bending of steering catheter 600. Accordingly, the rotational alignment of outer sheath 500 with steering catheter 600 is beneficial to the proper positioning of valve cover 550 and prosthetic mitral valve 100 relative to mitral valve annulus 158. As an alternative to the engagement of luer lock 532 within slot 142, outer sheath 500 may be rotationally aligned with and fixed relative to steering catheter 600 using any other key and corresponding keyway feature, slots and corresponding tabs, or other rotational keying mechanism known in the art. Alternatively or additionally, alignment markers may be provided at the proximal end of catheter assembly 400 to visually indicate rotational alignment.

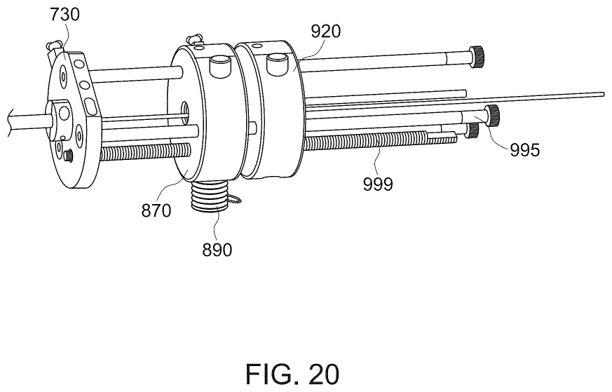

Extension catheter 700 extends proximally through steering catheter 600 and steering catheter handle 132 where its proximal end 702 may be fixedly coupled to an extension catheter holder 730 positioned proximally of the steering catheter handle. As shown in FIG. 20, extension catheter holder 730 may be in the form of a disk having a central aperture (not shown) that enables suture catheter 800 to extend proximally therethrough. A flush port (not shown) may be positioned on the rim of extension catheter holder 730 to allow for flushing the interior of extension catheter 700.

Suture catheter 800 extends proximally through extension catheter 700, steering catheter handle 132, and extension catheter holder 730 and may be fixedly coupled at its proximal end 804 to a suture catheter control 870 positioned proximally of the extension catheter holder. Suture catheter control 870 may be in the form of a disk or puck having a central aperture (not shown) through which nosecone catheter 900 may extend proximally. Although not shown in the figures, an internally threaded metal insert may be press fit into suture catheter control 870. The insert may have a circumferential groove or recess sized to receive a set screw for holding the insert in place. The threaded portion 805 at the proximal end 804 of suture catheter 800 may mate with the internal threads of the insert to connect the suture catheter to the suture catheter control. A flush port (not shown) may be positioned on the rim of suture catheter control 870 to allow for flushing the interior of suture catheter 800.

Nosecone catheter 900 extends proximally from nosecone 950 through suture catheter 800, steering catheter handle 132, extension catheter holder 730 and suture catheter control 870 and may be coupled to a nosecone catheter control 920. Nosecone catheter control 920 may be in the form of a disk or puck to which a proximal portion of nosecone catheter 900 may be fixedly attached. A central aperture (not shown) through nosecone catheter control 920 enables the proximal end 902 of nosecone catheter 900 and guidewire 975 to extend proximally of the nosecone catheter control. Although both suture catheter control 870 and nosecone catheter control 920 have been shown and described as disks or pucks, they may have different shapes, including spheres, ovoids, polygons or other shapes that may be grasped and translated. In addition, the shape of suture catheter control 870 may be the same as or different than the shape of nosecone catheter control 920.

Both suture catheter control 870 and nosecone catheter control 920 may be selectively locked in longitudinal positions relative to extension catheter holder 730 or may be released so that the suture catheter control is translatable proximally and distally relative to the extension catheter holder and the nosecone catheter control, and the nosecone catheter control is translatable proximally and distally relative to the extension catheter holder and the suture catheter control. In order to maintain the axial alignment of suture catheter control 870 and nosecone catheter control 920 with extension catheter holder 730 (and also the axial alignment of the components of catheter assembly 400), both the suture catheter control and the nosecone catheter control may translate along a plurality of rigid alignment rods 995. Each alignment rod 995 may be connected at its distal end to extension catheter holder 730 and may extend proximally therefrom through a plurality of apertures in suture catheter control 870 and another plurality of apertures in nosecone catheter control 920. An enlarged fitting 997 may be positioned on the free end of each alignment rod 995 to prevent nosecone catheter control 920 and suture catheter control 870 from being removed from the rods. Alignment rods 995 assure that suture catheter control 870 and nosecone catheter control 920 freely slide in the proximal and distal directions as handle assembly 300 is operated.

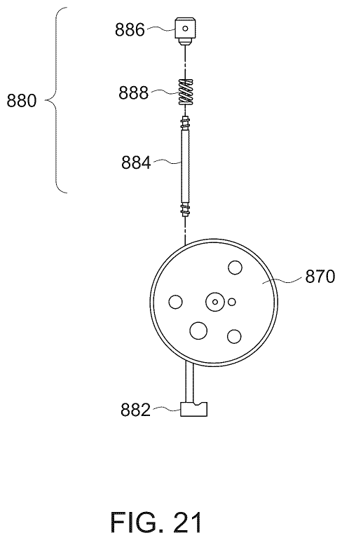

In one embodiment, suture catheter control 870 may include a release mechanism 880, while nosecone catheter control 920 may include a release mechanism 930. Release mechanisms 880 and 930 may have the same or similar structures, and may interact with a threaded rod or lead screw 999 that is fixedly connected at its distal end to extension catheter holder 730 and that extends proximally through apertures (not shown) in suture catheter control 870 and nosecone catheter control 920. As shown in the exploded view of FIG. 21, release mechanisms 880 and 930 may each include an end piece 882 engageable with threaded rod 999. A pin 884 may be connected at one end (such as by threaded engagement) to end piece 882 and may be connected at an opposite end (such as by threaded engagement) to a pushbutton 886. A spring 888 mounted on pin 884 between end piece 882 and pushbutton 886 may bias the end piece into engagement with threaded rod 999.

The engagement of release mechanism 880 with threaded rod 999 prevents suture catheter control 870 from translating proximally or distally relative to extension catheter holder 730 and nosecone catheter control 920. Depressing pushbutton 886 to disengage release mechanism 880 from threaded rod 999 frees suture catheter control 870 to translate along the threaded rod either proximally or distally relative to extension catheter holder 730 and nosecone catheter control 920. Once suture catheter control 870 is in the desired position, releasing release mechanism 880 will again bias end piece 882 into engagement with threaded rod 999, locking the suture catheter control in a fixed longitudinal position relative to the threaded rod. Moving suture catheter control 870 proximally or distally relative to nosecone catheter control 920 will selectively translate suture catheter 800 relative to nosecone catheter 900, nosecone 950 and the other components of catheter assembly 400. A set screw 890 on the rim of suture catheter control 870 opposite end piece 882 may be used during the loading of prosthetic mitral valve 100 in the catheter assembly. As explained more fully below, during valve loading, threaded rod 999 is turned to simultaneously retract suture catheter control 870 and nosecone catheter control 920. The forces generated during loading as the prosthetic mitral valve 100 is collapsed and pulled into valve cover 550 are so great that the force exerted by spring 888 may be overcome and release mechanism 880 may skip some threads on the threaded rod. In order to keep end piece 882 engaged with threaded rod 999 and prevent such skipping, set screw 890 may be screwed inwardly against the end piece, thereby enabling suture catheter control 870 to retract prosthetic mitral valve 100 smoothly and uniformly during the loading process. Although the use of a threaded rod 999 is shown, handle assembly 300 may include a rod having other structures (grooves, divots, depressions, etc.) for engaging with the end piece 882 of release mechanisms 880 and 930 when in the engaged position.

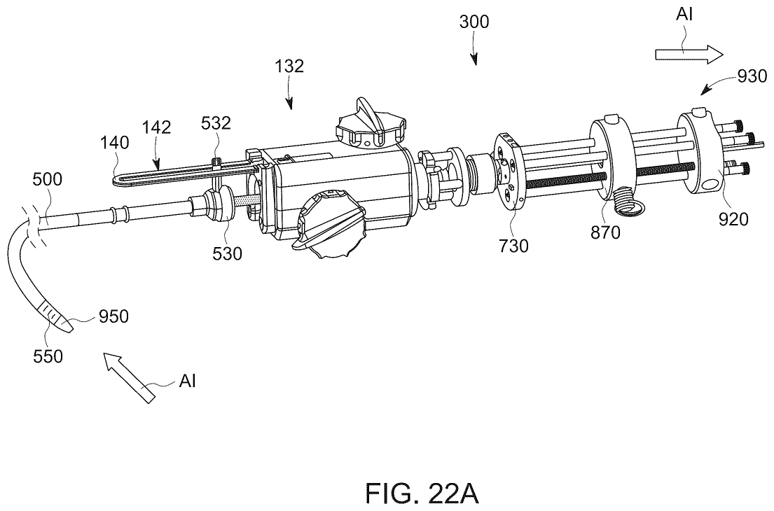

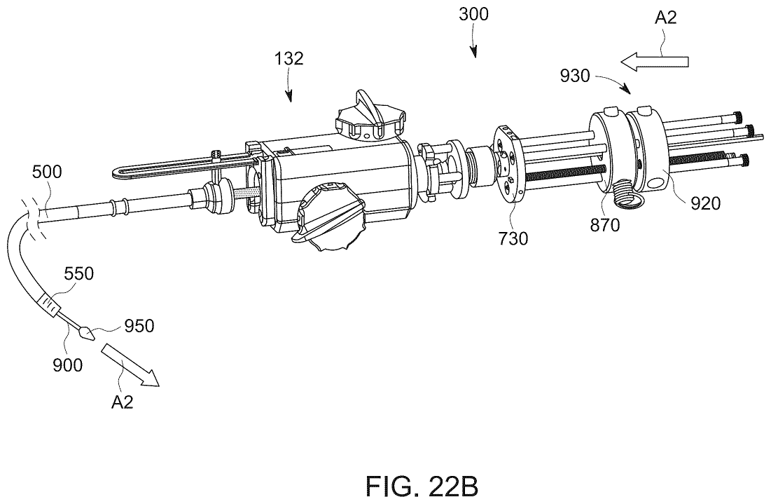

Similarly, release mechanism 930 may be biased into engagement with threaded rod 999 within nosecone catheter control 920. This engagement prevents nosecone catheter control 920 from translating proximally or distally relative to extension catheter holder 730 and suture catheter control 870. Depressing pushbutton 886 disengages release mechanism 930 from threaded rod 999, freeing nosecone catheter control 920 to translate along the threaded rod either proximally or distally relative to extension catheter holder 730 and suture catheter control 870. Moving nosecone catheter control 920 proximally or distally relative to suture catheter control 870 will selectively translate nosecone catheter 900 and nosecone 950 relative to suture catheter 800 and the other components of catheter assembly 400. Arrows A1 in FIG. 22A show the retraction of nosecone catheter control 920 and the corresponding retraction of nosecone catheter 900 and nosecone 950. Arrows A2 in FIG. 22B, on the other hand, show the advancement of nosecone catheter control 920 and the corresponding advancement of nosecone catheter 900 and nosecone 950. Once nosecone catheter control 920 is in the desired position, releasing release mechanism 930 will again bias end piece 882 into engagement with threaded rod 999, locking the nosecone catheter control in a fixed longitudinal position relative to the threaded rod. Although a spring-loaded pin such as shown has been found to provide effective and rapid actuation, other mechanisms for quickly disengaging suture catheter control 870 and nosecone catheter control 920 from threaded rod 999 may additionally or alternatively be utilized, such as a toggle release, snap shackle, quick-release skewer, and/or set screw, for example.

There are several advantages that result from having nosecone catheter 900 independently movable. When advancing suture catheter 800 to deploy prosthetic mitral valve 100, the suture catheter may not travel a sufficient distance before the advancing valve at the end of the suture catheter encounters nosecone 950. In such event, nosecone catheter 900 and nosecone 950 may be advanced distally by an additional amount to provide the space needed for suture catheter 800 to fully deploy the valve. Also, once prosthetic mitral valve 100 has been released, nosecone 950 is separated from valve cover 550 by the distance the prosthetic mitral valve had occupied, such as by about 40 mm. Following release of the prosthetic valve, catheter assembly 400 has to be pulled back and removed from the patient. By retracting nosecone 950 all the way against the open end of valve cover 550, any sharp edges on the various components of the catheter assembly can be secured within outer sheath 500 so as to not contact any surrounding tissue. Retracting nosecone 950 against valve cover 550 while in the left side of the heart may also avoid drawing any air that may be present in the catheter assembly into the right side of the heart (where there is relatively low pressure) as the catheter assembly is withdrawn.

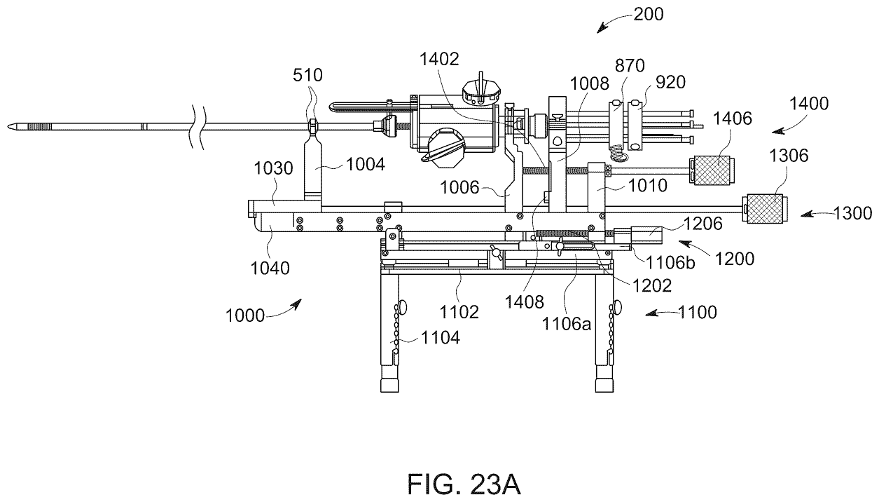

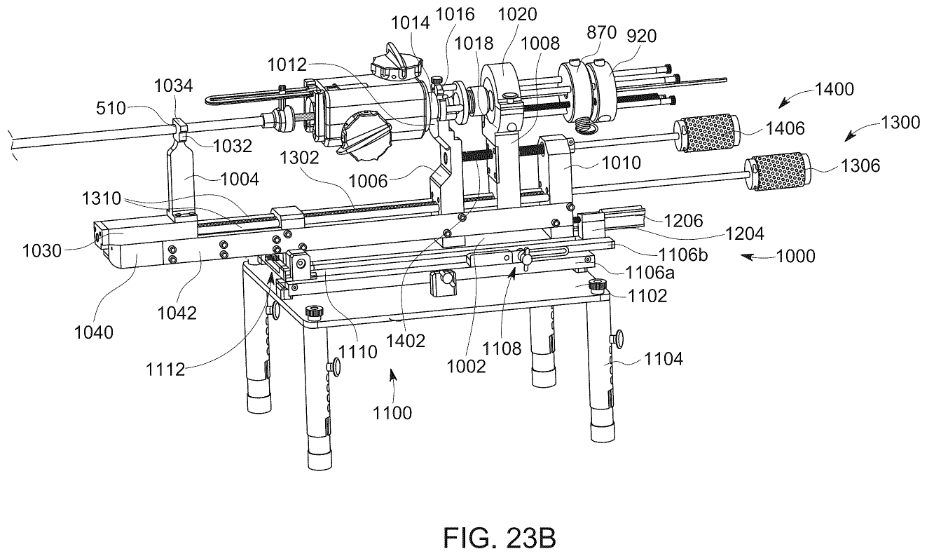

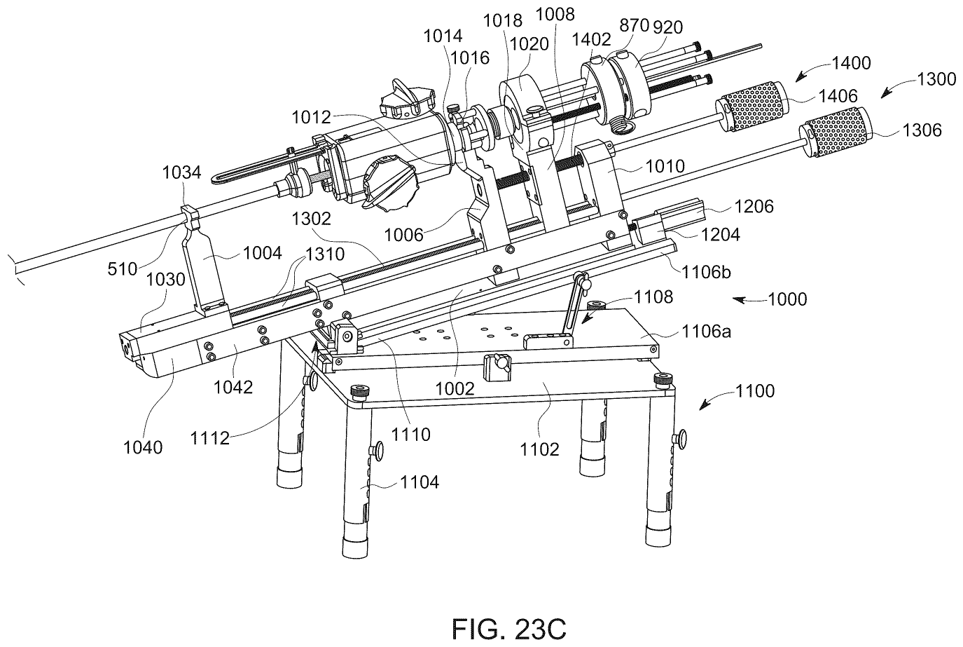

Referring to FIGS. 23A-23C, delivery system 200 may be supported by a stabilizer 1000 that includes a base 1002 and a plurality of rigid supports 1004, 1006, 1008 and 1010. A handle support 1006 may be fixedly connected to base 1002 and may extend upward to a semi-circular cradle 1012 that supports a disk-shaped member 1014 connected to the proximal end of steering catheter handle 132. A cover 1016 may be assembled to cradle 1012 over disk-shaped member 1014 to secure the disk-shaped member and steering catheter handle 132 to handle support 1006. Cradle 1012 and cover 1016 together support disk-shaped member 1014 in a manner that enables steering catheter handle 132 to be rotated about its longitudinal axis in both clockwise and counterclockwise directions. A support 1010 also may be fixedly connected to base 1002 and may extend upward therefrom to a height lower than that of handle support 1006. An extension catheter support 1008 between handle support 1006 and support 1010, however, may be spaced above and translatable relative to base 1002. Extension catheter support 1008 may extend upward to a semi-circular cradle 1018 having a recess sized and shaped to receive extension catheter holder 730. A cover 1020 may be assembled to cradle 1018 over extension catheter holder 730 to secure the extension catheter holder in its assembled position.

Outer sheath support 1004 may be fixedly mounted to a slider block 1030 near the distal end of base 1002 and may extend upward to a semi-circular cradle 1032 sized to receive outer sheath 500. Cradle 1032 has a width sized to be received between two rings 510 spaced from one another around the exterior of outer sheath 500. The engagement of cradle 1032 between rings 510 prevents outer sheath 500 from translating proximally or distally relative to outer sheath support 1004. A cover 1034 may be assembled to outer sheath support 1004 over outer sheath 500 to secure the outer sheath in its assembled position. Slider block 1030 may be positioned within a channel in the top surface of a slider block guide 1040. Slider block 1030 may be formed from anodized aluminum or another lightweight material, while slider block guide 1040 may be formed from polyoxymethylene or another low friction material so that the slider block slides smoothly and easily on the slider block guide. A pair of braces 1042 fixedly connected to the opposite sides of support 1010 and handle support 1006 may also connect to opposite sides of slider block guide 1040 to support the slider block guide below slider block 1030. As will be explained below, slider block 1030 may slide in slider block guide 1040 distally and proximally relative to base 1002 and delivery system 200 mounted thereon.

Stabilizer 1000 may be slidably mounted to a portable table 1100 that includes a platform 1102 supported by a plurality of height-adjustable legs 1104. A second platform 1106 may be slidably connected to platform 1102. Second platform 1106 may include a bottom plate 1106a and a top plate 1106b that are hingedly connected to one another at one end so that the proximal end of the top plate can be raised or lowered relative to the bottom plate and platform 1102, orienting delivery system 200 at a transverse angle to horizontal. A lock 1108 may secure top plate 1106b at a desired angular orientation relative to platform 1102. Top plate 1106b may have side rails 1110 that are undercut to define a pair of slots 1112 that extend the length of the top plate. Slots 1112 may be sized to receive the longitudinal edges of base 1002, enabling the base to slide proximally and distally relative to second platform 1106 and table 1100.

Stabilizer 1000 also includes a plurality of actuators that selectively control the translation of base 1002 and the translation of the components of catheter assembly 400 relative to one another. Base actuator 1200 may include a threaded rod or lead screw 1202 that is engaged with internal threads in support 1010. At its proximal end, lead screw 1202 may be coupled to a support post 1204 fixedly attached to second platform 1106. The coupling of lead screw 1202 to support post 1204 is such that the lead screw is rotatable but not translatable relative to support post 1204. An actuating knob 1206 may be affixed to the proximal end of lead screw 1202. Rotating actuating knob 1206 in one direction will cause base 1002 and all of the components assembled to the base to advance distally relative to second platform 1106 and table 1100, while rotating the actuating knob in the opposite direction will cause the base and the components assembled to the base to retract proximally relative to the second platform and the table.

An outer sheath actuator 1300 includes a rod 1302, at least a distal portion of which is threaded, supported for rotation through handle support 1006, extension catheter support 1008 and support 1010. The distal end of threaded rod or lead screw 1302 is journaled for rotation in slider block 1030, and an actuating knob 1306 may be affixed to its proximal end. Lead screw 1302 rotates freely within apertures in handle support 1006 and support 1010, while being engaged with threads within extension catheter support 1008 so that the lead screw rotates but does not translate relative to supports 1006 and 1010, but translates relative to support 1008. As a result, while extension catheter support 1008 is held in a fixed position (as explained below), rotation of actuating knob 1306 in one direction causes slider block 1030 to translate proximally, retracting outer sheath 500 relative to the other components of catheter assembly 400. Rotation of actuator knob 1306 in the opposite direction causes slider block 1030 to translate distally, advancing outer sheath 500 relative to the other components of catheter assembly 400. A pair of smooth, rigid rods 1310 may extend from slider block 1030 proximally through apertures in handle support 1006, extension catheter support 1008 and support 1010 to maintain the alignment of outer sheath support 1004 with the other portions of delivery system 200 as they translate relative to one another. The retraction of outer sheath 500 may be utilized to deploy a prosthetic heart valve held within valve cover 550 at the distal end of the outer sheath, while the advancement of the outer sheath/valve cover over the prosthetic heart valve may be utilized to recapture the valve.

Stabilizer 1000 also includes a valve positioning actuator 1400 that translates several of the components of catheter assembly 400 together relative to steering catheter 600. Positioning actuator 1400 includes a rod 1402, at least a distal portion of which is threaded. Threaded rod or lead screw 1402 is supported for rotation at its distal end in handle support 1006, extends through extension catheter support 1008, and is supported for rotation at its proximal end in support 1010. Positioning actuator 1400 is journaled in handle support 1006 and support 1010 so that it does not translate relative to those supports as it is rotated. However, lead screw 1402 is threadedly engaged with internal threads in extension catheter support 1008. As noted previously, extension catheter holder 730 is captured in the cradle 1018 in extension catheter support 1008, preventing the extension catheter holder from translating proximally or distally relative to the extension catheter support. Similarly, steering catheter handle 132 is supported by handle support 1006, which is held in a fixed position on base 1002. As a result of this arrangement, rotation of positioning actuator 1400 (via actuating knob 1406) results in the translation of extension catheter support 1008 relative to handle support 1006 and support 1010. The translation of extension catheter support 1008 results in the simultaneous translation of suture catheter control 870 and nosecone catheter control 920 by virtue of their connection to threaded rod 999, as well as the simultaneous translation of outer sheath 500 by virtue of its connection to lead screw 1302. However, when positioning actuator 1400 is not rotated, the connection of extension catheter support 1008 to lead screw 1402 holds the extension catheter support in a fixed position relative to handle support 1006 and support 1010 and prevents it from translating. A release mechanism 1408 may be incorporated on extension catheter support 1008 to release the extension catheter support from lead screw 1402, enabling the rapid manual translation of the extension catheter support as will be explained below.

In view of the forgoing connections among the various components, the rotation of positioning actuator 1400 in a first direction to translate extension catheter support 1008 simultaneously causes slider block 1030, extension catheter holder 730, suture catheter control 870 and nosecone catheter control 920 to retract proximally, while the position of steering catheter handle 132 remains fixed. Rotation of positioning actuator 1400 in the opposite direction causes the advancement in the distal direction of slider block 1030, extension catheter holder 730, suture catheter control 870 and nosecone catheter control 920, again without translating steering catheter handle 132.

As explained above, suture catheter control 870 may be translated proximally or distally relative to the other components of catheter assembly 400 by depressing release mechanism 880 and manually sliding the suture catheter control proximally or distally. Translation of suture catheter control 870 causes suture catheter 800 to translate relative to each of outer sheath 500, steering catheter 600, extension catheter 700, nosecone catheter 900 and nosecone 950. As will be explained below, the proximal retraction of suture catheter 800 relative to extension catheter 700 and/or outer sheath 500 increases the axial tension on tethers 850 to keep them connected to prosthetic mitral valve 100 and to maintain the prosthetic valve in a pre-deployed position within delivery system 200, while the distal advancement of the suture catheter relative to the extension catheter and/or the outer sheath releases the axial tension on the tethers and allows deployment of the prosthetic valve.

Similarly, nosecone catheter control 920 may be translated proximally or distally relative to the other components of catheter assembly 400 by depressing release mechanism 930 and manually sliding the nosecone catheter control proximally or distally. Translation of nosecone catheter control 920 causes the simultaneous translation of nosecone catheter 900 and nosecone 950 relative to outer sheath 500, steering catheter 600, extension catheter 700 and suture catheter 800.

The following will describe the loading of prosthetic mitral valve 100 into the valve cover 550 of delivery system 200. To begin, nosecone 950 is removed from nosecone catheter 900 and the various components of catheter assembly 400 are flushed to remove air from the system. Flushing is accomplished by attaching flush lines to outer sheath hemostasis valve 530, and to the steering catheter hemostasis valve, extension catheter flush port, suture catheter flush port and to a hemostasis valve (not shown) attached to the proximal end of nosecone catheter 900. A flush plug (not shown) may be attached to the distal end of valve cover 550 and a heparinized saline solution may be fed under pressure through the flush plug to valve cover 550 and the various components of catheter assembly 400. Stopcocks at the proximal ends of the flush lines may be selectively opened and closed to control the flow of the saline solution through the components of the catheter assembly both individually and collectively until all or substantially all air bubbles have been removed therefrom. Vibration may be applied to catheter assembly 400 to facilitate the removal of air from the system. To confirm proper deairing, after the flush plug has been removed from valve cover 550, steering catheter 600 may be advanced until its distal end is exposed beyond the distal end of the valve cover, extension catheter 700 may be advanced until its distal end is exposed beyond the distal end of the steering catheter, suture catheter 800 may be advanced until its distal end is exposed beyond the distal end of the extension catheter, and nosecone catheter 900 may be advanced until its distal end is exposed beyond the distal end of the suture catheter, all while these portions are submerged in a saline bath. Any air bubbles evident on any of these components should be removed using conventional techniques. Methods for flushing catheter assembly 400 are described in U.S. Patent Publication No. 2018/0126095, the disclosure of which is hereby incorporated by reference herein.

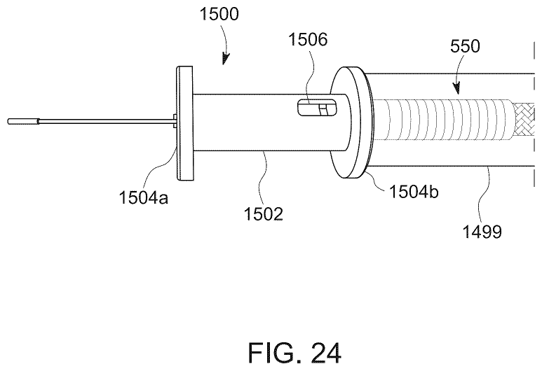

Following the deairing procedure, the components of catheter assembly 400 should be positioned for the loading of prosthetic mitral valve 100 into valve cover 550. To do this according to one embodiment, nosecone catheter 900 is retracted until its distal end is just within suture catheter 800, the suture catheter and the nosecone catheter are retracted together until their distal ends are within the can 720 at the distal end of extension catheter 700, and the extension catheter is retracted until can 720 contacts the distal end of steering catheter 600. Outer sheath 500 may then be advanced until can 720 is positioned near the midpoint of valve cover 550. A loading tube 1499 may then be assembled over outer sheath 500 so that it extends from outer sheath support 1004 to the distal end of valve cover 550. With the distal end of valve cover 550 protruding from the loading tube, a loading funnel 1500 is assembled to the valve cover. If at this point tantalum ring 558 is already assembled to the distal end of valve cover 550, it is removed therefrom to enable the assembly of loading funnel 1500 to the valve cover. Loading funnel 1500 may be formed from titanium (although other sufficiently strong and nonreactive materials may be used) and has a cylindrical body 1502 with a lumen therethrough and flanges 1504a, 1504b on respective ends of the body, as shown in FIG. 24. Internal threads at the proximal end of loading funnel 1500 mate with the external threads at the distal end of valve cover 550, enabling the loading funnel to be screwed onto the end of the valve cover. An elongated window 1506 in the body 1502 of loading funnel 1500 enables the user to see the interior of the loading funnel as prosthetic mitral valve 100 is retracted into and through the loading funnel. Although a threaded connection of loading funnel 1500 to valve cover 550 has been described, any other removable connection may be used, such as a snap fit connection, bayonet connection, clamping connection, and the like. Once loading funnel 1500 has been assembled to valve cover 550, loading tube 1499 may be adjusted to increase its length until one end of the loading tube presses against the flange 1504b at the proximal end of the loading funnel and the other end of the loading tube presses against outer sheath support 1004, thereby applying tension to outer sheath 500.

Subsequently, outer sheath actuator 1300 may be rotated to retract outer sheath 500 proximally until can 720 is positioned within the window 1506 of loading funnel 1500. Suture catheter 800 may then be advanced by depressing release mechanism 880 and translating suture catheter control 870 distally until the distal end of the suture catheter is accessible at the distal end of loading funnel 1500. Prior to attaching prosthetic mitral valve 100 to suture catheter 800, the cuff of the prosthetic valve may be pleated inwardly to facilitate the retraction of the prosthetic valve into valve cover 550 and prevent the cuff from becoming damaged as the prosthetic valve is retracted. With the distal end of suture catheter 800 exposed beyond the end of loading funnel 1500, and with the loading funnel and prosthetic mitral valve assembly 865 submerged within a saline bath, the mitral valve assembly may be assembled to the suture catheter. The entire prosthetic mitral valve assembly 865 is attached to suture catheter 800 by screwing distal suture ring 830 to the proximal suture ring 820 at the distal end of the suture catheter. Release mechanism 880 may again be depressed and suture catheter control 870 may be retracted proximally to retract suture catheter 800 and prosthetic mitral valve assembly 865 until the atrial petals 114 of prosthetic mitral valve 100 are within the lumen of loading funnel 1500. Packaging assembly 867 may then be removed from prosthetic mitral valve 100.

At this juncture, release mechanism 930 may be depressed and nosecone catheter control 920 may be advanced distally until it abuts suture catheter control 870 to advance nosecone catheter 900 through prosthetic mitral valve 100 until it is exposed distally of the valve. A thin, elongated balloon mounted on a hypotube (not shown) that is internally threaded at its proximal end may then be inflated with saline and secured by threaded engagement to the distal end of nosecone catheter 900. With the balloon hypotube attached to nosecone catheter 900, release mechanism 930 may again be depressed and nosecone catheter control 920 may be retracted proximally to retract the balloon hypotube to a position in which the balloon is positioned within prosthetic mitral valve 100. A balloon mandrel (not shown) having an enlarged distal end (larger than the inner diameter of the balloon hypotube) may be inserted longitudinally through the balloon hypotube and nosecone catheter 900 until it protrudes from the proximal end of the nosecone catheter, with the enlarged distal end contacting the distal end of the balloon hypotube. An additional hypotube (not shown) may be assembled over the proximal end of the balloon mandrel proximally of the proximal end of nosecone catheter 900. This additional hypotube may have a screw at its proximal end that may be turned to extend the length of the balloon mandrel. A clamp (not shown) may then be applied to the proximal end of the balloon mandrel, proximally of the additional hypotube. With the clamp locked to the balloon mandrel, the screw may be turned to lengthen the additional hypotube, putting the balloon mandrel under tension. Together, the contact of the enlarged distal end of the balloon mandrel with the distal end of the hypotube and the clamp attached to the proximal end of the balloon mandrel prevent the balloon hypotube from being squeezed out distally as prosthetic mitral valve 100 is collapsed and retracted into loading funnel 1500.

As a next step, threaded rod 999 may be turned through the use of a removable handle (not shown), thereby retracting suture catheter 800 and nosecone catheter 900 together relative to extension catheter 700, steering catheter 600 and outer sheath 500. The retraction of suture catheter 800 and nosecone catheter 900 will draw prosthetic mitral valve 100 into loading funnel 1500 until atrial petals 114 are fully within can 720 at the distal end of extension catheter 700 as will be visible through window 1506 in the loading funnel. Subsequently, actuating knob 1306 on lead screw 1302 may be rotated to advance outer sheath 500 distally, whereby prosthetic mitral valve 100 continues to collapse as loading funnel 1500 is advanced over it. When the prosthetic valve is fully within loading funnel 1500, the balloon within the collapsed valve may be deflated. Actuating knob 1306 may be used to continue rotating lead screw 1302 such that the collapsed prosthetic valve will be transferred from loading funnel 1500 into the advancing valve cover 550. The collapsed prosthetic valve will be fully transferred to valve cover 550 when can 720 is visible at the two most proximal laser cuts in the valve cover.

With prosthetic mitral valve 100 fully loaded in valve cover 550, loading tube 1499 may be adjusted to decrease its length and remove the compressive forces exerted on it. The loading tube may then be pulled back until valve cover 550 can be grasped and held in place, enabling loading funnel 1500 to be removed, followed by the loading tube. The balloon clamp and the additional hypotube may then be removed from the proximal end of the balloon mandrel and the balloon hypotube may be pulled forward along with nosecone catheter 900 until the distal end of the nosecone catheter is exposed beyond valve cover 550. The balloon hypotube may then be detached from nosecone catheter 900 along with the balloon mandrel, tantalum ring 558 may be threaded onto the distal end of valve cover 550, and nosecone 950 may be threaded onto the distal end of the nosecone catheter. Nosecone catheter 900 may then be retracted through operation of nosecone catheter control 920 until nosecone 950 seats against the distal end of valve cover 550.

In a preferred arrangement, suture catheter 800 and tethers 850 are pretensioned after prosthetic mitral valve 100 has been loaded into valve cover 550 and prior to the insertion of catheter assembly 400 into the patient. To pretension suture catheter 800 and tethers 850, a torque wrench can be assembled to the proximal end of threaded rod 999 and the rod can be turned to retract the suture catheter relative to extension catheter 700 until a desired tension in the suture catheter is achieved. More particularly, once the can 720 at the distal end of extension catheter 700 is seated against the tip ring 620 of steering catheter 600, any further tension applied to suture catheter 800 will be transferred to the steering catheter. Applying further tension to suture catheter 800 (through further rotation of threaded rod 999) will cause the suture catheter to lengthen slightly by the separation of the laser cuts at its distal end, creating a spring-like tension in the suture catheter. The desired tension should be within a range that is sufficient to hold prosthetic mitral valve 100 securely within can 720 at the distal end of extension catheter 700 and to maintain the connection of tethers 850 to the pins 118 on the prosthetic valve, but not so high as to significantly decrease the flexibility of the distal end of catheter assembly 400. The tension/compression relationship between suture catheter 800 and extension catheter 700 should be maintained until the atrial petals 114 of prosthetic mitral valve 100 have been released from extension catheter can 720. If the tension in suture catheter 800 is too low or if the tension is not maintained long enough, atrial petals 114 may be partially released too soon. Another reason for a defined pre-tensioning is that during deflection of the most distal section of catheter assembly 400, the catheter components that are located further from the neutral line will foreshorten more that those closer to the neutral line. Pre-tensioning of suture catheter 800 will prevent the suture catheter, which is closer to the neutral line, from losing the required tension. Delivery system 200 is now ready for delivering prosthetic mitral valve 100 into the left atrium LA of the patient and deploying the prosthetic valve in mitral valve annulus 158.

In a conventional procedure, an incision is made in the patient's groin to access the femoral vein, and a guidewire, such as guidewire 975, is fed up through the femoral vein, advanced through the inferior vena cava 150 to the right atrium RA, through a puncture in intra-atrial septum 154 and into the left atrium LA. Catheter assembly 400 is then advanced over guidewire 975 until nosecone 950 is located in left atrium LA confronting the mitral valve annulus 158. At this juncture, balloon 570 may be inflated around valve cover 550, as described above. While visualizing balloon 570 with ultrasound imaging, catheter assembly 400 is advanced further until the balloon is aligned within mitral valve annulus 158.

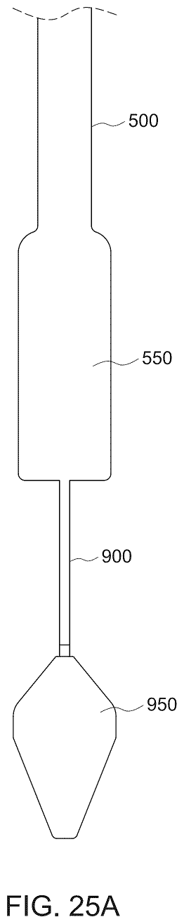

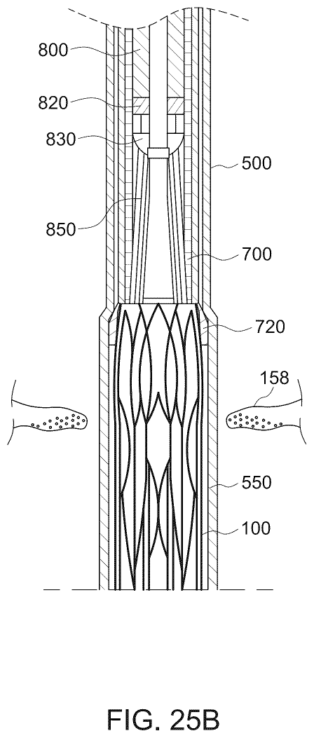

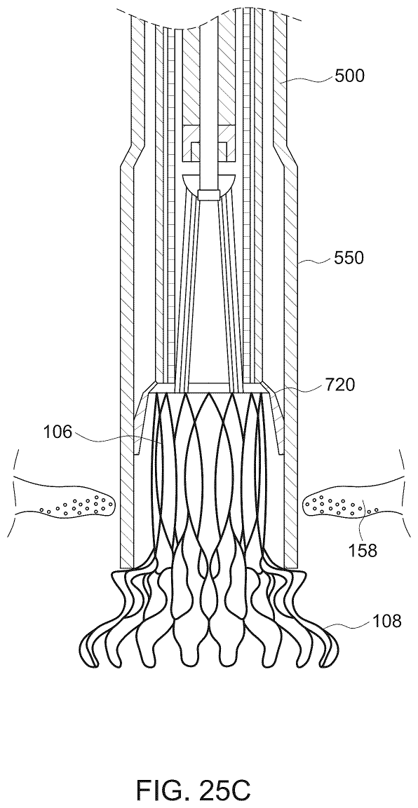

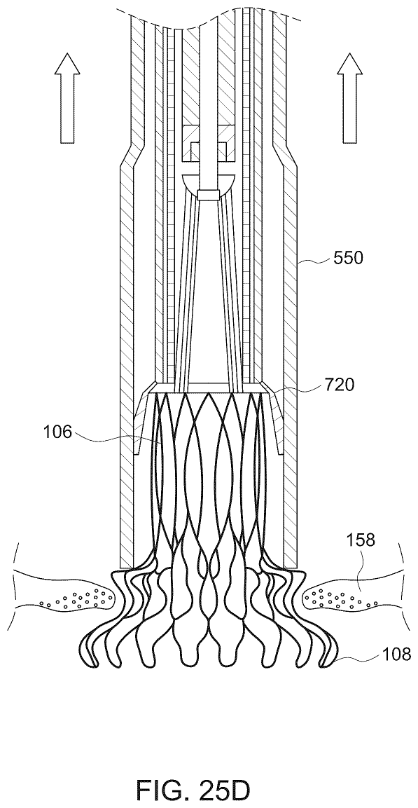

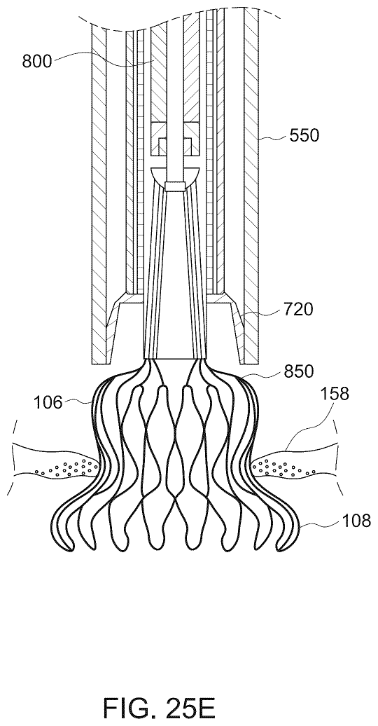

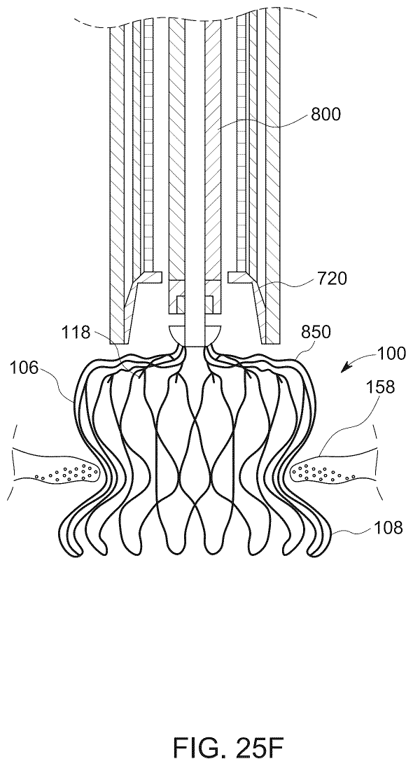

FIGS. 25A-25F schematically illustrate the deployment and release of prosthetic mitral valve 100 at mitral valve annulus 158. Once catheter assembly 400 is properly positioned, outer sheath 500 and valve cover 550 may be retracted proximally by rotating outer sheath actuator 1300 while nosecone 950 remains in place, thereby creating a separation between the nosecone and the valve cover. Outer sheath 500 and valve cover 550 are retracted by an amount that provides sufficient space for the deployment of prosthetic mitral valve 100, as shown in FIG. 25A. (Although mitral valve 100 will be fully exposed and expanded when nosecone 950 and valve cover 550 are in the relative positions shown in FIG. 25A, the mitral valve has been omitted from this figure to more clearly show the other elements.) FIG. 25B is a longitudinal cross-sectional view of catheter assembly 400 in position at mitral valve annulus 158, with valve cover 550 positioned so that inflated balloon 570 (not shown in the figure) is aligned within the annulus, a distal portion of prosthetic mitral valve 100 is positioned on the ventricular side of the annulus, and a proximal portion of the prosthetic mitral valve is positioned on the atrial side of the annulus. Outer sheath 500 and valve cover 550 may then be retracted further, as shown in FIG. 25C, by rotation of outer sheath actuator 1300, enabling the ventricular anchor 108 of prosthetic mitral valve 100 to be released and expand within left ventricle LV. As shown in FIGS. 25D and 25E, following the release and expansion of ventricular anchor 108, outer sheath 500 and valve cover 550 may be further retracted to fully expose prosthetic mitral valve 100 (i.e., atrial anchor 106), while maintaining tension in tethers 850. Once, fully exposed, prosthetic mitral valve 100 may be retracted proximally to bring the ventricular anchor into contact against the ventricular side of mitral valve annulus 158. This may be accomplished by rotating positioning actuator 1400 to retract extension catheter 700 and suture catheter 800. If extension catheter 700 is already in a fully retracted position, ventricular anchor 108 may be brought into contact with the ventricular side of mitral valve annulus 158 by rotating prosthetic mitral valve 100. This may be done by rotating steering catheter handle 132 in the posterior direction. As a result of the bends at the distal end of catheter assembly 400, rotating steering catheter handle 132 will cause prosthetic mitral valve 100 to pivot relative to the plane of mitral valve annulus 158 until ventricular anchor 108 contacts the mitral valve annulus. Ventricular anchor 108 will deflect as it contacts mitral valve annulus 158, enabling prosthetic mitral valve 100 to move closer to its proper orientation relative to the valve annulus, with atrial anchor 106 on the atrial side of mitral valve annulus 158.

At this point, prosthetic mitral valve 100 may still be held by tethers 850 in a not yet fully deployed condition, with atrial anchor 106 still held in a collapsed condition within the can 720 of extension catheter 700. Since leaflets 113 are not yet functioning when ventricular anchor 108 is retracted against mitral valve annulus 158, the positioning of the ventricular anchor will impede the flow of blood from left atrium LA to left ventricle LV. Therefore, as ventricular anchor 108 is retracted against mitral valve annulus 158, it is desirable to simultaneously advance suture catheter 800 to release atrial anchor 106 so that blood can begin flowing through the prosthetic valve. This may be accomplished by actuating release mechanism 1408 to quickly retract extension catheter support 1008 and extension catheter 700, drawing ventricular anchor 108 against the mitral valve annulus and, at the same time, actuating release mechanism 880 to translate suture catheter control 870 and suture catheter 800 distally to relieve the tension in tethers 850, enabling atrial anchor 106 to expand and release from can 720, as shown in FIG. 25F. In this regard, the distance between suture catheter control 870 (in its pre-deployed position) and extension catheter holder 730 enables suture catheter 800 to move distally a sufficient distance to reliably cause tethers 850 to move forward, invert, slide off of pins 118 and detach from atrial anchor 106. The longitudinal position of nosecone 950 relative to suture catheter 800 can be adjusted if additional space is needed while the suture catheter is being advanced. Upon its expansion, atrial anchor 106 will contact the atrial side of mitral valve annulus 158, thus sandwiching the mitral valve annulus between the atrial anchor and the ventricular anchor and securing prosthetic mitral valve 100 in place.

The ability to rapidly actuate and translate suture catheter control 870 enables atrial anchor 106 to be deployed quickly and prosthetic mitral valve 100 to begin operating quickly, restoring blood flow from left atrium LA to left ventricle LV. After prosthetic mitral valve 100 has been detached from suture catheter 800, the suture catheter may be retracted by again depressing release mechanism 880 and translating suture catheter control 870 proximally away from extension catheter holder 730, pulling tethers 850 back into extension catheter 700. Nosecone catheter 900 may then be retracted by depressing release mechanism 930 and translating nosecone catheter control 920 proximally until nosecone 950 is seated against the distal end of valve cover 550. Subsequently, balloon 570 may be deflated, collapsing inflation/deflation lumen 572, and catheter assembly 400 may be removed from the patient.

During the deployment of prosthetic mitral valve 100, situations may arise in which it becomes desirable to re-sheathe the prosthetic valve and either reposition it or remove it entirely from the patient. This may occur when prosthetic mitral valve 100 is not properly positioned or not properly oriented in the native mitral annulus, or when the deployed prosthetic valve has been damaged or otherwise is not functioning as intended. During a re-sheathing procedure, however, substantial compressive forces are exerted on outer sheath 500 as prosthetic mitral valve 100 is collapsed and retracted into valve cover 550 or as the outer sheath is advanced to push the valve cover over the prosthetic valve. For example, when prosthetic mitral valve 100 is first loaded into valve cover 550, the loading is carried out at room temperature (below the transition temperature of nitinol where nitinol is more flexible) with the use of loading funnel 1500, and catheter assembly 400 is straight. Even under these conditions, the loading force generated could be about 10-100 lbs. or more, and more likely about 20-60 lbs. During re-sheathing in the body (without loading funnel 1500 and above the transition temperature of nitinol), on the other hand, the loading forces generated could be much higher, not only because the nitinol will be above its transition temperature and therefore stiffer, but also because the distal end of the catheter assembly will be deflected to a substantial degree. To achieve high compressive forces at the distal end of catheter assembly 400 can be very challenging as the catheter assembly is up to 6 ft. long and its distal end is deflected in two planes. Although the coil/braid portion 514/516 at the distal end of outer sheath 500 provides flexibility while permitting deployment of prosthetic mitral valve 100, that structure does not provide sufficient compressive strength for re-sheathing. As compressive forces are exerted on the coil/braid portion 514/516 during re-sheathing, the large gaps between adjacent coils will collapse, causing a foreshortening of the coil/braid portion by 50%-70% or more as the individual coils contact one another. Since braided sleeve 516 is fixedly connected at both ends to coiled layer 514, the braided sleeve must shorten by the same amount, which is either impossible or will cause the sleeve to enlarge in diameter substantially.

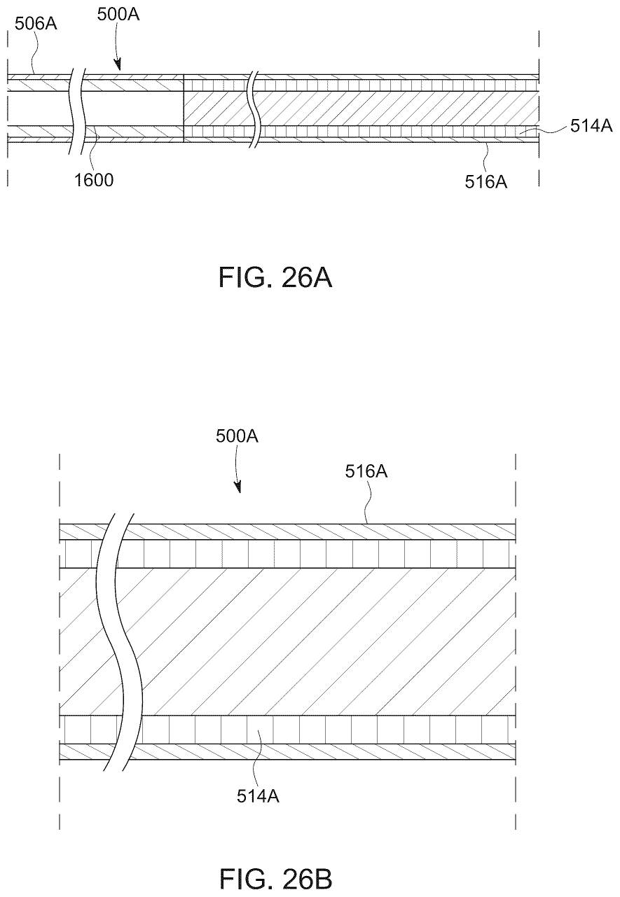

One approach to facilitate the re-sheathing of a fully or partially deployed prosthetic mitral valve 100 is to decouple the unsheathing and re-sheathing forces by detaching braided sleeve 516 from one end of coiled layer 514. The distal end of an alternate embodiment of an outer sheath 500A incorporating this decoupling concept is shown in the longitudinal cross-section of FIG. 26A. Outer sheath 500A is substantially the same as outer sheath 500 described above. However, in outer sheath 500A, the proximal end of coiled layer 514A is not connected to the proximal end of braided sleeve 516A. Rather, where catheter assembly 400 includes outer sheath 500A, an additional hypotube 1600 is concentrically arranged between the outer sheath and steering catheter 600, and the proximal end of coiled layer 514A is fixedly connected by laser welding to the distal end of the hypotube. Hypotube 1600 extends proximally within outer sheath 500A to a proximal end positioned proximally of steering catheter handle 132. A hypotube control (not shown) attached to the proximal end of hypotube 1600 enables the hypotube to be translated proximally and distally relative to the other components of catheter assembly 400. Hypotube 1600 may include a plurality of circumferential laser cut slits along its length similar to those of the proximal portion 506A of outer sheath 500A so that the hypotube does not impede the bending of the proximal portion of the outer sheath. Additionally, the inner diameter of outer sheath 500A is sufficiently large that hypotube 1600 is able to freely slide proximally and distally within the outer sheath.

The distal end of braided sleeve 516A is fixedly connected to the distal end of coiled layer 514A as described above for outer sheath 500, and the proximal end of the braided sleeve is connected to the proximal portion 506A of outer sheath 500A, also as described above for outer sheath 500. However, as the proximal end of coiled layer 514A is not connected to the proximal end of braided sleeve 516A or to outer sheath 500A, the proximal end of the coiled layer is able to move proximally and distally within braided sleeve 516A.

During the deployment of prosthetic mitral valve 100 as described above, outer sheath 500A and hypotube 1600 may be retracted together relative to the other components of catheter assembly 400 to deploy the prosthetic valve from the distal end of valve cover 550. In a preferred arrangement, the hypotube control may be releasably coupled to outer sheath support 1004 so that rotation of outer sheath actuator 1300 simultaneously translates outer sheath 500A and hypotube 1600. One approach to accomplish this may be for the hypotube control to have a disk or puck shape that is similar to those of nosecone catheter control 920 and suture catheter control 870. A separate sheath support bracket (not shown) may be connected to and project laterally from outer sheath support 1004, with the hypotube control and the sheath support bracket connected to one another by a threaded rod. A release mechanism (not shown), similar to release mechanisms 880 and 930, may be mounted in the hypotube control (or the sheath support bracket). This arrangement will operate in substantially the same manner as the arrangement described above by which nosecone catheter control 920 is releasably coupled to suture catheter control 870. That is, when the release mechanism is not depressed, hypotube 1600 and outer sheath 500A will be coupled to one another and will translate together. However, depressing the release mechanism will decouple hypotube 1600 from outer sheath 500A so that they can be translated independently from one another. As outer sheath 500A is retracted simultaneously with hypotube 1600, braided sleeve 516A will lengthen along with coiled layer 514A, collapsing the braided sleeve around the coiled layer to maintain the alignment of the individual coils with one another and prevent the coiled layer from deforming to an oval shape. Prosthetic mitral valve 100 will thus be deployed in the same manner as described above.

During a re-sheathing procedure, on the other hand, valve cover 550 is advanced distally until it collapses and covers prosthetic mitral valve 100. To advance valve cover 550 distally, a compressive longitudinal force must be applied to the valve cover. This force is applied by advancing coiled layer 514A, whose distal end is attached to valve cover 550. Coiled layer 514A is advanced by decoupling the hypotube control from outer sheath actuator 1300 and translating hypotube 1600 distally. As valve cover 550 contacts and begins to collapse prosthetic mitral valve 100, friction and the forces required to collapse the prosthetic valve will prevent the valve cover from easily sliding over the prosthetic valve. As a result, a compressive force will be applied to coiled layer 514A, causing the individual coils to collapse against one another in a stacked relationship, shown in FIG. 26B, shortening the coiled layer. Since the hypotube control is not coupled to the outer sheath actuator 1300 as hypotube 1600 is translated, the hypotube is able to move distally relative to outer sheath 500A, and braided sleeve 516A does not collapse along with coiled layer 514A. However, once the coils of coiled layer 514A become stacked, continued distal advancement of hypotube 1600 will cause the stacked coiled layer to advance distally, pushing valve cover 550 toward and over prosthetic mitral valve 100. Since the proximal end of braided sleeve 516A is not connected to coiled layer 514A, the braided sleeve will not collapse, permitting the coils of the coiled layer to move freely relative to one another.

As noted above, the distal end of catheter assembly 400 may be maneuvered into a compound curve shape in order to properly orient prosthetic mitral valve 100 for deployment. As also noted above, during a re-sheathing procedure, the coils of coiled layer 514A collapse as hypotube 1600 is advanced distally, shortening the coiled layer. Coiled layer 514A may be foreshortened by up to 50%-70% or more of its original length. As coiled layer 514A shortens, hypotube 1600 will advance into braided sleeve 516A. However, hypotube 1600 is not as flexible as coiled layer 514A, such that the advancement of the hypotube could cause the compound curve shape at the distal end of catheter assembly 400 to straighten and valve cover 550 to become misaligned with the deployed prosthetic valve. In order to avoid this occurrence, it is preferable to form coiled layer 514A with a sufficient length that, when the coils of the coiled layer are stacked against one another, the proximal end of the coiled layer will be aligned at or near the proximal end of braided sleeve 516A. With such arrangement, hypotube 1600 will not enter braided sleeve 516A as it is advanced, and therefore will not interfere with the compound bends formed at the distal end of catheter assembly 400.

The foregoing arrangement facilitates the re-sheathing of a deployed or partially deployed prosthetic heart valve by decoupling the unsheathing and re-sheathing forces. During a re-sheathing procedure, the decoupling of the proximal end of braided sleeve 516A from coiled layer 514A enables the individual coils to move freely relative to one another such that they become stacked and the full compressive force generated by the advancement of hypotube 1600 can be transmitted through the coiled layer to valve cover 550, enabling the valve cover to collapse and cover the prosthetic valve.

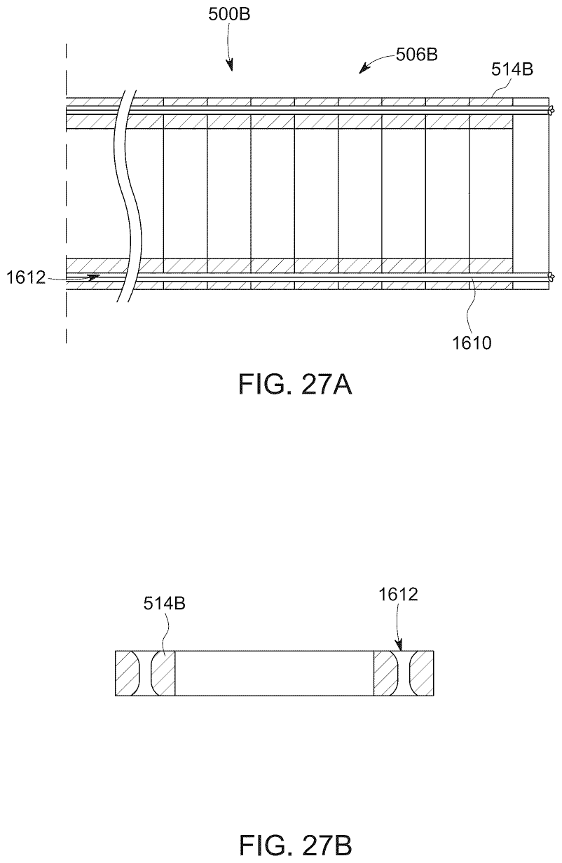

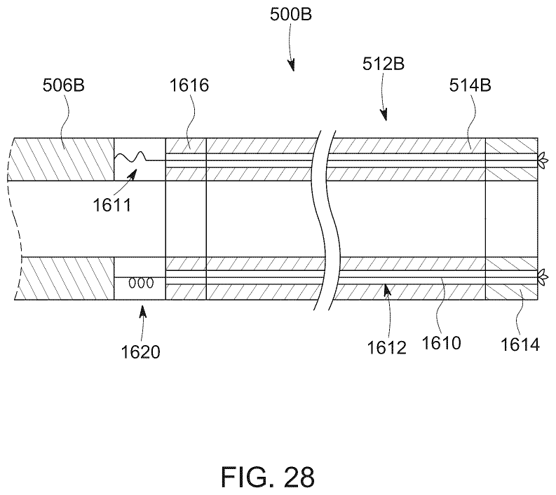

In a different embodiment, coiled layer 514A may be replaced with a series of individual rings that may be held in coaxial alignment by a plurality of filaments threaded through each of the rings from a proximal end of the series of rings to its distal end. FIG. 27A is a longitudinal cross-section of the distal end of an outer sheath 500B. Outer sheath 500B is similar to outer sheath 500A described above. Like outer sheath 500A, outer sheath 500B includes an inner element having a distal end connected to valve cover 550 and a proximal end connected to the distal end of the proximal portion 506B of the outer sheath. However, rather than a coiled element, such as coiled layer 514A, the inner element of this embodiment may have a stacked arrangement of individual rings 514B formed from medical grade stainless steel or from a durable polymer, such as polyether ether ketone (PEEK), for example using an injection molding process. However, unlike in outer sheath 500A, ring stack 514B may not be covered by a braided sleeve. Rather, ring stack 514B includes a plurality of thin wires or filaments 1610, each of which extends through a series of aligned apertures 1612 formed in the individual rings. Filaments 1610 may be wires formed from stainless steel, nitinol or another metal, threads of suture material or a polymer, and the like. Preferably, the material forming filaments 1610 has a degree of elasticity so as to not impede the deflection of the rings in stack 514B relative to one another. Apertures 1612 may be formed by drilling through each of the rings in stack 514B using a laser, preferably a short pulse laser, which will drill apertures having a very smooth sidewall. Rather than a short pulse laser, however, other types of lasers may be used, followed by a secondary process, such as e-polishing, to smooth the surface. As shown in FIG. 27B, the ends of apertures 1612 optionally may be outwardly tapered to minimize any drag on filaments 1610 and any sharp edges that could potentially damage the filaments, particularly when the rings in stack 514B are significantly deflected relative to one another. So that filaments 1610 can maintain the alignment of the individual rings, the diameter of apertures 1612 is preferably only slightly larger than the diameter of the filaments. For example, for filaments 1610 having a diameter of about 0.006 in., apertures 1612 may have a diameter of about 0.007 in. Ring stack 514B may have at least two series of apertures 1612 to maintain the coaxial alignment of the individual rings. However, ring stack 514B may include three or more series of apertures 1612 as needed to keep the individual rings from becoming offset from one another. The series of apertures 1612 may be spaced apart around the circumference of ring stack 514B. For example, two series of apertures 1612 may be spaced apart by about 180°, three series of apertures may be spaced apart by about 120°, etc. In the event that filaments 1610 are too weak to withstand the very high unsheathing forces experienced by outer sheath 500B, the outer sheath may include a braided sleeve that surrounds ring stack 514B to help withstand those forces.

The distal ends of filaments 1610 may be welded or otherwise connected to one or more rings 1614 at the distal end of ring stack 514B. After threading through apertures 1612, the proximal ends of filaments 1610 may be welded or otherwise connected to the proximal portion 506B of outer sheath 500B. To enable the proximal ends of filaments 1610 to pass through the ring 1616 at the proximal end of ring stack 514B, the proximal portion 506B of outer sheath 500B may include a series of slots 1620 that extend proximally from the distal end of the proximal portion to a closed end. Each filament 1610 may extend through a respective one of slots 1620 until its proximal end is connected to the closed end of the slot. For example, for a filament 1610 formed from metal wire, the proximal end of the filament may be welded to the closed of a slot 1620.

When filaments 1610 are formed from an elastic material, such as a polymer or nitinol, they may have sufficient elasticity to lengthen slightly when placed under tension, such as when certain filaments are placed under tension as the flexible portion 512B of outer sheath 500B is deflected to align with mitral valve annulus 158. However, when formed from a nonelastic material, each filament 1610 may include a proximal end portion 1611 in which the filament is bent to have a zig-zag shape, a sinusoidal shape, a coiled shape or some other shape that will enable the filament to lengthen slightly when subjected to such tension. Preferably, any lengthening of filaments 1610 is elastic, such that the filaments return to their original length when they are no longer under tension. That will enable filaments 1610 to maintain the alignment of the rings in stack 514B.

To summarize the foregoing, the present disclosure describes a delivery system for delivering a medical device to a targeted anatomical site within a patient. The delivery system includes a catheter assembly, including an outer sheath having a proximal end and a distal end; a valve cover at the distal end of the outer sheath, the valve cover having a proximal end, a distal end, and a size and shape for housing the medical device in a collapsed condition; a balloon disposed circumferentially about an exterior surface of the valve cover between the proximal end and the distal end of the valve cover, the balloon having an inflated condition and a deflated condition; and a plurality of catheters coaxially arranged within the outer sheath and slidable in proximal and distal directions relative to the outer sheath; and/or
the delivery system may further include a polymer layer surrounding the valve cover, the balloon being disposed between the exterior surface of the valve cover and the polymer layer; and/or
the delivery system may further include an inflation/deflation lumen having a tube proximal end connectable to an inflation medium, a tube distal end connected to the balloon, and a tube lumen defined between the tube proximal end and the tube distal end, the inflation/deflation lumen having a collapsed condition in which the tube lumen is collapsed with one side of the inflation/deflation lumen contacting an opposite side of the inflation/deflation lumen, and an expanded condition in which the one side of the inflation/deflation lumen is spaced apart from the opposite side of the inflation/deflation lumen to form an open lumen to the balloon; and/or
the inflation/deflation lumen may be formed from a collapsible polymer film; and/or
the delivery system may further include a first polymer layer surrounding the outer sheath, the inflation/deflation lumen being disposed between an exterior surface of the outer sheath and the first polymer layer; and/or
the delivery system may further include a second polymer layer surrounding the valve cover, the balloon being disposed between the exterior surface of the valve cover and the second polymer layer; and/or
the first polymer layer may be integral with the second polymer layer; and/or
the balloon in the inflated condition may define a toroid circumscribing the valve cover; and/or
the delivery system may further include a base; and a handle assembly mounted to the base for controlling movement of the outer sheath and the plurality of catheters relative to one another.

The present disclosure also describes another delivery system for delivering a medical device to a targeted anatomical site within a patient. The delivery system includes a catheter assembly extending in a longitudinal direction, the catheter assembly including an outer sheath having a first portion and a second portion that is more flexible than the first portion, the second portion having a coiled layer and a braided sleeve disposed around the coiled layer, the coiled layer having a proximal end and a distal end, and the braided sleeve having a proximal end connected to the first portion of the outer sheath and a distal end connected to the distal end of the coiled layer; a hypotube disposed within the first portion of the outer sheath, a distal end of the hypotube being connected to the proximal end of the coiled layer; a valve cover connected to the distal end of the coiled layer, the valve cover having a proximal end, a distal end, and a size and shape for housing the medical device in a collapsed condition; and a plurality of catheters coaxially arranged within the outer sheath and slidable in proximal and distal directions relative to the outer sheath; and/or
the delivery system may further include a polymer layer surrounding the valve cover and the first and second portions of the outer sheath; and/or
the delivery system may further include a balloon disposed circumferentially about an exterior surface of the valve cover between the proximal end and the distal end of the valve cover, the balloon having an inflated condition and a deflated condition; and/or
the braided sleeve may have an initial length and a tensioned length, and the coiled layer may have an initial length and a compressed length, the compressed length of the coiled layer being at least as long as the initial length of the braided sleeve; and/or
the outer sheath may be operably couplable to the hypotube such that, when the hypotube is coupled to the outer sheath, translation of the outer sheath in the longitudinal direction causes simultaneous translation of the hypotube in the longitudinal direction, and when the hypotube is decoupled from the outer sheath, the hypotube can translate in the longitudinal direction independently of the outer sheath; and/or
the outer sheath may further include a base; and a handle assembly mounted to the base for controlling movement of the outer sheath and the plurality of catheters relative to one another.

The present disclosure describes yet another delivery system for delivering a medical device to a targeted anatomical site within a patient. The delivery system includes a catheter assembly, including an outer sheath extending in a longitudinal direction and having a first portion and a second portion that is more flexible than the first portion, the second portion having a plurality of rings arranged adjacent one another in a stack, the stack having a proximal end and a distal end, the proximal end being connected to a distal end of the first portion; a plurality of filaments extending through each of the plurality of rings, each filament having a distal end connected to the distal end of the ring stack and a proximal end connected to the first portion of the outer sheath; a valve cover at the distal end of the ring stack, the valve cover having a size and shape for housing the medical device in a collapsed condition; and a plurality of catheters coaxially arranged within the outer sheath and slidable in proximal and distal directions relative to the outer sheath; and/or
the first portion may include a plurality of slots extending in the longitudinal direction, each slot having an open end at the distal end of the first portion and a closed end spaced proximally of the open end, and the proximal end of each of the filaments may be connected within a respective one of the slots; and/or
each of the filaments may extend in the longitudinal direction, a proximal section of each of the filaments having a filament length oriented in directions transverse to the longitudinal direction; and/or
the delivery system may further include a polymer layer surrounding the valve cover and the first and second portions of the outer sheath; and/or
the delivery system may further include a balloon disposed circumferentially about an exterior surface of the valve cover between the proximal end and the distal end of the valve cover, the balloon having an inflated condition and a deflated condition; and/or
the delivery system may further include a base; and a handle assembly mounted to the base for controlling movement of the outer sheath and the plurality of catheters relative to one another.

The present disclosure describes a still further delivery system for delivering a medical device to a targeted anatomical site within a patient. The delivery system includes a catheter assembly having a longitudinal axis, the catheter assembly including an outer sheath having a proximal end and a distal end; a valve cover at the distal end of the outer sheath, the valve cover having a proximal end, a distal end, and a size and a shape for housing the medical device in a collapsed condition; a balloon disposed circumferentially about an exterior surface of the valve cover between the proximal end and the distal end of the valve cover, the balloon having an inflated condition and a deflated condition; an extension catheter disposed within the outer sheath and slidable in proximal and distal directions relative to the outer sheath; and a suture catheter disposed within the extension catheter and slidable in the proximal and distal directions relative to the extension catheter, the suture catheter being adapted to maintain a connection to the medical device until deployment of the medical device; a base; and a handle assembly mounted to the base for controlling movement of the outer sheath, the extension catheter and the suture catheter relative to one another, the handle assembly including an outer sheath actuator operatively connected to the outer sheath and operable to move the outer sheath in the proximal and distal directions relative to the base; an extension catheter holder connected to the extension catheter; a suture catheter control connected to the suture catheter proximally of the extension catheter holder; and a locking mechanism having a locked condition in which the position of the suture catheter control is fixed relative to the extension catheter holder so that the suture catheter and the extension catheter move together in the proximal and distal directions relative to the outer sheath and having a release condition in which the suture catheter control is movable in the proximal and distal directions relative to the extension catheter holder.

The present disclosure describes yet another delivery system for delivering a medical device to a targeted anatomical site within a patient. The delivery system includes a catheter assembly having a longitudinal axis, the catheter assembly including an outer sheath having a first portion and a second portion that is more flexible than the first portion, the second portion having a coiled layer and a braided sleeve disposed around the coiled layer, the coiled layer having a proximal end and a distal end, and the braided sleeve having a proximal end connected to the first portion of the outer sheath and a distal end connected to the distal end of the coiled layer; a hypotube disposed within the first portion of the outer sheath, a distal end of the hypotube being connected to the proximal end of the coiled layer; a valve cover connected to the distal end of the coiled layer, the valve cover having a proximal end, a distal end, and a size and shape for housing the medical device in a collapsed condition; an extension catheter disposed within the outer sheath and slidable in proximal and distal directions relative to the outer sheath; and a suture catheter disposed within the extension catheter and slidable in the proximal and distal directions relative to the extension catheter, the suture catheter being adapted to maintain a connection to the medical device until deployment of the medical device; a base; and a handle assembly mounted to the base for controlling movement of the outer sheath, the extension catheter and the suture catheter relative to one another, the handle assembly including an outer sheath actuator operatively connected to the outer sheath and operable to move the outer sheath in the proximal and distal directions relative to the base; an extension catheter holder connected to the extension catheter; a suture catheter control connected to the suture catheter proximally of the extension catheter holder; and a locking mechanism having a locked condition in which the position of the suture catheter control is fixed relative to the extension catheter holder so that the suture catheter and the extension catheter move together in the proximal and distal directions relative to the outer sheath and having a release condition in which the suture catheter control is movable in the proximal and distal directions relative to the extension catheter holder.

The present disclosure describes still another delivery system for delivering a medical device to a targeted anatomical site within a patient. The delivery system includes a catheter assembly, including an outer sheath extending in a longitudinal direction and having a first portion and a second portion that is more flexible than the first portion, the second portion having a plurality of rings arranged adjacent one another in a stack, the stack having a proximal end and a distal end, the proximal end being connected to a distal end of the first portion; a plurality of filaments extending through each of the plurality of rings, each filament having a distal end connected to the distal end of the ring stack and a proximal end connected to the first portion of the outer sheath; a valve cover at the distal end of the ring stack, the valve cover having a size and shape for housing the medical device in a collapsed condition; an extension catheter disposed within the outer sheath and slidable in proximal and distal directions relative to the outer sheath; and a suture catheter disposed within the extension catheter and slidable in the proximal and distal directions relative to the extension catheter, the suture catheter being adapted to maintain a connection to the medical device until deployment of the medical device; a base; and a handle assembly mounted to the base for controlling movement of the outer sheath, the extension catheter and the suture catheter relative to one another, the handle assembly including an outer sheath actuator operatively connected to the outer sheath and operable to move the outer sheath in the proximal and distal directions relative to the base; an extension catheter holder connected to the extension catheter; a suture catheter control connected to the suture catheter proximally of the extension catheter holder; and a locking mechanism having a locked condition in which the position of the suture catheter control is fixed relative to the extension catheter holder so that the suture catheter and the extension catheter move together in the proximal and distal directions relative to the outer sheath and having a release condition in which the suture catheter control is movable in the proximal and distal directions relative to the extension catheter holder.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims. For example, although the delivery system has been described herein for use to deliver and deploy a prosthetic mitral valve, the delivery system may also be used to deliver and deploy a different prosthetic heart valve, such as a prosthetic aortic valve, tricuspid valve or pulmonary valve, or another implantable medical device.

1. A delivery system for delivering a medical device to a targeted anatomical site within a patient, the delivery system comprising:
a catheter assembly, including:
an outer sheath having a proximal end and a distal end;
a valve cover at the distal end of the outer sheath, the valve cover having a proximal end, a distal end, and a size and a shape for housing the medical device in a collapsed condition;
a balloon disposed circumferentially about an exterior surface of the valve cover between the proximal end and the distal end of the valve cover, the balloon having an inflated condition and a deflated condition; and
a plurality of catheters coaxially arranged within the outer sheath and slidable in proximal and distal directions relative to the outer sheath.

2. The delivery system as claimed in claim 1, further comprising a polymer layer surrounding the valve cover, the balloon being disposed between the exterior surface of the valve cover and the polymer layer.

3. The delivery system as claimed in claim 1, further comprising an inflation/deflation lumen having a tube proximal end connectable to an inflation medium, a tube distal end connected to the balloon, and a tube lumen defined between the tube proximal end and the tube distal end, the inflation/deflation lumen having a collapsed condition in which the tube lumen is collapsed with one side of the inflation/deflation lumen contacting an opposite side of the inflation/deflation lumen, and an expanded condition in which the one side of the inflation/deflation lumen is spaced apart from the opposite side of the inflation/deflation lumen to form an open lumen to the balloon.

4. The delivery system as claimed in claim 3, wherein the inflation/deflation lumen is formed from a collapsible polymer film.

5. The delivery system as claimed in claim 3, further comprising a first polymer layer surrounding the outer sheath, the inflation/deflation lumen being disposed between an exterior surface of the outer sheath and the first polymer layer.

6. The delivery system as claimed in claim 5, further comprising a second polymer layer surrounding the valve cover, the balloon being disposed between the exterior surface of the valve cover and the second polymer layer.

7. The delivery system as claimed in claim 6, wherein the first polymer layer is integral with the second polymer layer.

8. The delivery system as claimed in claim 1, wherein the balloon in the inflated condition defines a toroid circumscribing the valve cover.

9. The delivery system as claimed in claim 1, further comprising:
a base; and
a handle assembly mounted to the base for controlling movement of the outer sheath and the plurality of catheters relative to one another.

10. A delivery system for delivering a medical device to a targeted anatomical site within a patient, the delivery system comprising:
a catheter assembly extending in a longitudinal direction, the catheter assembly including:
an outer sheath having a first portion and a second portion that is more flexible than the first portion, the second portion having a coiled layer and a braided sleeve disposed around the coiled layer, the coiled layer having a proximal end and a distal end, and the braided sleeve having a proximal end connected to the first portion of the outer sheath and a distal end connected to the distal end of the coiled layer;
a hypotube disposed within the first portion of the outer sheath, a distal end of the hypotube being connected to the proximal end of the coiled layer;
a valve cover connected to the distal end of the coiled layer, the valve cover having a proximal end, a distal end, and a size and a shape for housing the medical device in a collapsed condition; and
a plurality of catheters coaxially arranged within the outer sheath and slidable in proximal and distal directions relative to the outer sheath.

11. The delivery system as claimed in claim 10, further comprising a polymer layer surrounding the valve cover and the first and second portions of the outer sheath.

12. The delivery system as claimed in claim 10, further comprising a balloon disposed circumferentially about an exterior surface of the valve cover between the proximal end and the distal end of the valve cover, the balloon having an inflated condition and a deflated condition.

13. The delivery system as claimed in claim 10, wherein the braided sleeve has an initial length and a tensioned length and the coiled layer has an initial length and a compressed length, the compressed length of the coiled layer being at least as long as the initial length of the braided sleeve.

14. The delivery system as claimed in claim 10, wherein the outer sheath is operably couplable to the hypotube such that, when the hypotube is coupled to the outer sheath, translation of the outer sheath in the longitudinal direction causes simultaneous translation of the hypotube in the longitudinal direction, and when the hypotube is decoupled from the outer sheath, the hypotube can translate in the longitudinal direction independently of the outer sheath.

15. The delivery system as claimed in claim 10, further comprising:
a base; and
a handle assembly mounted to the base for controlling movement of the outer sheath, the hypotube and the plurality of catheters relative to one another.

16. A delivery system for delivering a medical device to a targeted anatomical site within a patient, the delivery system comprising:
a catheter assembly, including:
an outer sheath extending in a longitudinal direction and having a first portion and a second portion that is more flexible than the first portion, the second portion having a plurality of rings arranged adjacent one another in a stack, the stack having a proximal end and a distal end, the proximal end being connected to a distal end of the first portion;
a plurality of filaments extending through each of the plurality of rings, each filament having a distal end connected to the distal end of the ring stack and a proximal end connected to the first portion of the outer sheath;
a valve cover at the distal end of the ring stack, the valve cover having a size and a shape for housing the medical device in a collapsed condition; and
a plurality of catheters coaxially arranged within the outer sheath and slidable in proximal and distal directions relative to the outer sheath.

17. The delivery system as claimed in claim 16, wherein the first portion includes a plurality of slots extending in the longitudinal direction, each slot having an open end at the distal end of the first portion and a closed end spaced proximally of the open end, and the proximal end of each of the filaments is connected within a respective one of the slots.

18. The delivery system as claimed in claim 16, wherein each of the filaments extends in the longitudinal direction, a proximal section of each of the filaments having a filament length oriented in directions transverse to the longitudinal direction.

19. The delivery system as claimed in claim 16, further comprising a polymer layer surrounding the valve cover and the first and second portions of the outer sheath.

20. The delivery system as claimed in claim 16, further comprising a balloon disposed circumferentially about an exterior surface of the valve cover between the proximal end and the distal end of the valve cover, the balloon having an inflated condition and a deflated condition.
